(19) United States
(12) Patent Application Publication
(10) Pub. No.: US 2026/0369403 A1
(43) Pub. Date: Nov. 19, 2026

(54) COMPUTER-IMPLEMENTED METHODS AND SYSTEMS FOR PERSONALIZED, EVIDENCE-INFORMED DIAGNOSTIC AND TREATMENT GUIDANCE

(71) Applicant: 1290161 B.C. LTD., Vancouver (CA)

(72) Inventors: Diane McIntosh, Vancouver (CA); Carol Mason, Richmond (CA); Vanessa Bois, Quebec City (CA); Oscar Medina, New Westminster (CA); Christopher Patrick, Ottawa (CA); Deborah Durrell, Nepean (CA); Brad Jobe, Toronto (CA); Robert Correia, Kinburn (CA); Bradley Chruszcz, Cambridge (CA)

(73) Assignee: 1290161 B.C. LTD., Vancouver, BC (CA)

(21) Appl. No.: 19/137,628

(22) PCT Filed: Feb. 28, 2023
(86) PCT No.: PCT/CA2023/050254

§ 371 Date: Jun. 10, 2025

Related U.S. Application Data
(60) Provisional application No. 63/432,895, filed on Dec. 15, 2022.

(51) Int. Cl. G16H 50/20; G16H 15/00; G16H 20/10

(52) U.S. Cl. CPC G16H 50/20; G16H 15/00; G16H 20/10

(57) ABSTRACT

A computer-implemented method, a device, and a computer readable storage medium are disclosed to provide personalized diagnostic and treatment guidance for a patient exhibiting one or more health condition symptoms. The method is performed by one or more processors and includes obtaining patient profile data that includes a result of evaluation test(s) or interview(s) of the patient; determining a diagnosis based on the patient profile data; determining, based on the patient profile data and the diagnosis of the patient, a personalized treatment plan to address a health disorder identified in the diagnosis of the patient; and generating an electronic report that includes an indication of the diagnosis and the personalized treatment plan. The personalized treatment plan includes a pharmacotherapy component identifying an ordered listing of pharmacological treatment options based on patient profile data and the diagnosis of the patient.

### CROSS-REFERENCE TO RELATED APPLICATION

This application claims priority to U.S. Provisional Application No. 63/432,895, filed on Dec. 15, 2022, the entire content of which is hereby expressly incorporated herein by reference.

### FIELD

The disclosed teachings relate generally to techniques for assessing and treating individuals. More particularly, the disclosed teachings relate to a computer-implemented platform that can provide personalized, evidence informed diagnostic and treatment guidance for diagnosing and treating patients.

### BACKGROUND

Successful patient outcomes rely on accurate diagnosis and treatments. A healthcare professional typically gathers a patient's history and assesses a patient's symptoms as well as recommend medical diagnostic tests for suspected diseases, disorders or other adverse health conditions. These diagnostic tests may include evidence based questionnaires/scales, physical examinations, and/or laboratory tests. Based on the patient data collected, the results of the medically recognized diagnostic tests, the use of recognized guidelines for diagnosing diseases as well as the health care professionals knowledge and experience, the healthcare professional may then diagnose the patient with a specific known disease or condition and prescribe known treatments.

For example, in the context of mental health, the diagnostic tests used for diagnosing and treating a mental health disorder may involve having a patient complete one or more broad questionnaires that include questions about the patient's behavioral symptoms. The patient's answers may be processed and compared to a list of broad, clinically significant categories including mental health disorders. The patient may then be asked to complete extensive behavioral questionnaire(s) targeted to mental health disorders. The patient's answers to the targeted questionnaire(s) may then be analyzed and used as input to diagnose the patient with a specific mental health disorder. Treatments may then be prescribed to the patient based on the diagnosis.

However, conventional methods for diagnosing and treating patients rely heavily upon the professional expertise and experience of the healthcare professional. Unfortunately, misdiagnoses are not uncommon by general practitioners for diseases and conditions in more specialized areas of medicine such as mental health. It is impossible for general healthcare professionals (HCP) who must have broad knowledge about the majority of health conditions to also have depth of knowledge in all areas of medical specialization. As a result, general HCPs must often rely on their own partial knowledge and experience about a possible disease or condition to make a diagnosis which is too often based on just the patient's medical profile and symptoms. HCP's too often do not pursue recognized diagnostic tests due to lack of time or lack of awareness of the existence and/or importance of these tests to arrive at an accurate diagnosis. As a consequence, the conventional diagnosis and treatment method is often performed iteratively until a suitable diagnosis and treatment are identified. Moreover, even if a patient is properly diagnosed by existing methods, treating the patient typically remains a trial and error process that involves applying treatments that are deemed suitable for the diagnosis, especially in complex areas such as mental health. This is further compounded by the fact that some patients respond well to certain treatments while other patient's with the same diagnosis fail to experience successful outcomes on the same treatment.

These challenges associated with conventional methods for diagnosing and treating patients are amplified further in the context of diagnosing and treating patients in areas of healthcare in which access to qualified and experienced healthcare professionals is limited or unavailable. In such scenarios, lack of access to qualified healthcare professionals in certain specialized disciplines, such as clinical psychiatrists for the diagnosis and treatment of mental health disorders, often means that either consultation with a qualified specialist is significantly delayed and/or that initial diagnoses and treatments are provided by a general practitioner, such as the patient's family doctor, who may not be equipped to accurately diagnose and treat health conditions in specialized areas of mental or physical health.

For example, in the context of mental health, due to poor access to psychiatric care, patients can wait up to 2 years for a specialist appointment. Evidence shows that delays in receiving an accurate diagnosis and thus, effective treatment, is associated with disease progression, reduced response to treatment, greater functional impairment, worsening physical health and heightened mortality.

General practitioners (GP) must have broad knowledge about how to diagnose and treat many health conditions. Even though well-established best-practice guidelines exist for diagnosing and treating many illnesses, GPs often do not have the depth of expertise to accurately diagnose and treat illnesses in more specialized areas of medicine but are often forced to in order to support their patients due to the lengthy wait time to see a medical specialist. In the case of mental illnesses, for example, it can be especially onerous for the GP to sift through the extensive guidelines to make the correct choices that meet the needs of each patient. In many specialty areas, including mental health, the research and guidelines are constantly being updated which makes it additionally difficult for GPs to stay up to date across many specialty areas.

To illustrate this point, the DSM-5 diagnosis of major depressive disorder can present in nearly 1000 different ways, there are more than a dozen evidence-based assessment scales to diagnose depression, there are 25 antidepressants recommended by the Canadian treatment guidelines for depression (CANMAT), and each patient has unique needs, with respect to factors such as acceptable side effects. Ensuring the right diagnosis and choosing the right treatment, personalized to a patient's needs, is more difficult in psychiatry than other medical specialties, because of the lack of objective tests, such as laboratory markers or radiological tests.

Despite strong empirical evidence and years of guideline recommendations, most GPs and psychiatrists do not use evidence-based assessment tools. As a result, more than half of all mental illnesses are initially misdiagnosed. Bipolar disorder is initially misdiagnosed more than 90% of the time, taking up to 10 years and assessments from three or four clinicians to receive an accurate diagnosis.

Given the high rate of misdiagnosis, it is not surprising that the current approaches to treatment of mental health disorders are often ineffective. For example, research shows that 70% of patients exhibiting symptoms of a mental health disorder/condition do not respond to their first prescribed treatment, and 30% still will not be in remission after their fourth treatment trial.

Moreover, evidence shows delays and inaccurate diagnosis can lead to disease progression, comorbidities, and poor treatment response. For example, major depressive disorder (MDD) detection and treatment rates are low in primary care settings, and treatment quality is often suboptimal in terms of follow-up and monitoring. However, timely treatment of MDD is critical, as findings from longitudinal studies suggest that the disorder increases the risk of disability, diabetes, cancer, heart disease, obesity, and cognitive impairment. In MDD, longer duration of no-treatment interval is an effective predictor of a poorer response to antidepressant treatment, a lower rate of remission, a higher risk of chronicity, and a higher number of recurrences. Early intervention is also crucial in bipolar disorder (BD) to improve patients' prognosis, possibly reversing cognitive deficits. Moreover, pharmacological and non-pharmacological treatments might work best in the earliest illness stages. Until mental illnesses are diagnosed and treated accurately, patients will not recover as quickly or as fully as they otherwise could.

Accordingly, there is a need for improved methods and systems for providing personalized, evidenced-informed diagnostic and treatment guidance for accurately diagnosing and effectively treating patients to manage symptoms of illness, restore functioning where possible and improve quality of life.

### SUMMARY

The present disclosure describes computer-implemented methods that deliver fast diagnostic and treatment verification and guidance using a consistent, standardized process to complete a patient assessment. For example, the disclosed computer-implemented methods could be used to validate an initial diagnosis of a mental health disorder by a non-psychiatric physician or to identify and correct an instance where such an initial diagnosis or treatment plan may be sub-optimal, using validated assessment tools and empirically validated treatment choices, which can lead to a faster recovery and lower the risk of relapse or progression to long-term disability.

Accordingly, a first aspect of the present disclosure provides a computer-implemented method for generating personalized diagnostic and treatment guidance. The method is executed by one or more processors. According to the method, patient profile data for a patient is obtained. The patient profile data may include a result of at least one evaluation test or interview of the patient, for example. A diagnosis of the patient may be determined based on the patient profile data. For example, the diagnosis of the patient may identify a health disorder of the patient. A personalized treatment plan to address the health disorder of the patient may be determined based on the patient profile data and the diagnosis of the patient. For example, the personalized treatment plan may include a pharmacotherapy component identifying an ordered listing of pharmacological treatment options, each pharmacological treatment option corresponding to a different pharmaceutical product for treatment of the health disorder. For example, ordering of the pharmacological treatment options in the ordered listing may be based on a ranking of the pharmacological treatment options for treatment of the health disorder based on the patient profile data and the diagnosis of the patient. An electronic report may be generated that includes an indication of the diagnosis of the patient, and an indication of the personalized treatment plan to address the health disorder of the patient.

In some embodiments of the method according to the first aspect of the present disclosure, each pharmacological treatment option corresponds to a different pharmaceutical product approved or commonly used off-label by medical specialists for treatment of the health disorder.

In some embodiments of the method according to the first aspect of the present disclosure, the ordered listing of pharmacological treatment options includes only a partial subset of the highest ranked pharmacological treatment options corresponding to pharmaceutical products approved or commonly used off-label by medical specialists for treatment of the health disorder.

In some embodiments of the method according to the first aspect of the present disclosure, the pharmacotherapy component of the personalized treatment plan for the patient includes pharmacological treatment guidance providing at least one of the following: guidance for starting a pharmacological treatment option from among the ordered listing of pharmacological treatment options; guidance for stopping a current pharmacological treatment option identified in the patient profile data; guidance for optimizing a dosage of a current pharmacological treatment option identified in the patient profile data; guidance for switching from a current pharmacological treatment option to a different pharmacological treatment option from among the ordered listing of pharmacological treatment options; and guidance for augmenting a current pharmacological treatment option with another different pharmacological treatment option from among the ordered listing of pharmacological treatment options.

In some embodiments of the method according to the first aspect of the present disclosure, determining the personalized treatment plan based on the patient profile data and the diagnostic assessment of the patient is performed by the one or more processors utilizing a rules engine to process the patient profile data and the diagnostic assessment relative to a rule set.

In some embodiments of the method according to the first aspect of the present disclosure, the rule set is configured to implement empirically validated treatment guidance.

In some embodiments of the method according to the first aspect of the present disclosure: the health disorder of the patient is a primary health disorder of the patient, the diagnosis identifying a second comorbid health disorder of the patient; and the personalized treatment plan addresses treatment of the primary health disorder and treatment of the second comorbid health disorder of the patient.

In some embodiments of the method according to the first aspect of the present disclosure, the pharmacotherapy component of the personalized treatment plan comprises: a first ordered listing of pharmacological treatment options for treatment of the primary health disorder, each pharmacological treatment option of the first ordered listing corresponding to a different pharmaceutical product for treatment of the primary health disorder, wherein the ordering of the pharmacological treatment options in the first ordered listing is based on a ranking of the pharmacological treatment options for treatment of the primary health disorder based on the patient profile data and the diagnosis of the patient; and a second ordered listing of pharmacological treatment options for treatment of the second comorbid health disorder, each pharmacological treatment option of the second ordered listing corresponding to a different pharmaceutical product for treatment of the second comorbid health disorder.

In some embodiments of the method according to the first aspect of the present disclosure, the ordering of the pharmacological treatment options in the second ordered listing is based on a ranking of the pharmacological treatment options for treatment of the second comorbid health disorder based on the patient profile data and the diagnosis of the patient.

In some embodiments of the method according to the first aspect of the present disclosure, the ordering of the pharmacological treatment options for treatment of the primary health disorder in the first ordered listing considers both treatment of the primary health disorder and treatment of the second comorbid disorder.

In some embodiments of the method according to the first aspect of the present disclosure, the ordering of the pharmacological treatment options for treatment of the secondary comorbid health disorder in the second ordered listing considers both treatment of the secondary comorbid health disorder and treatment of the primary health disorder.

In some embodiments of the method according to the first aspect of the present disclosure, determining the diagnosis of the patient comprises: identifying multiple health disorders of the patient based on the patient profile data; identifying one health disorder of the multiple health disorders of the patient as the primary health disorder of the patient based on a hierarchy for treatment of health disorders; identifying another health disorder of the multiple health disorders of the patient as the second comorbid health disorder of the patient based on the hierarchy for treatment of health disorders.

In some embodiments of the method according to the first aspect of the present disclosure, determining the diagnosis of the patient further comprises identifying each remaining health disorder of the multiple health disorders of the patient as a further comorbidity to the primary health disorder based on the hierarchy for treatment of health disorders.

In some embodiments of the method according to the first aspect of the present disclosure, the personalized treatment plan addresses treatment of the primary health disorder and treatment of the health disorders identified as comorbidities to the primary health disorder in accordance with a priority based on the hierarchy for treatment of health disorders.

In some embodiments of the method according to the first aspect of the present disclosure, the ranking of the pharmacological treatment options is further based on a respective tolerability rating for each pharmacological treatment option.

In some embodiments of the method according to the first aspect of the present disclosure, for each pharmacological treatment option, the respective tolerability rating is a single rating that is a function of respective ratings for a plurality of tolerability factors for that pharmacological treatment option.

In some embodiments of the method according to the first aspect of the present disclosure, the plurality of tolerability factors for a given pharmacological treatment option correspond to potential side effects from an associated pharmacological treatment class, and wherein the respective rating for a given tolerability factor indicates a likelihood of experiencing the side effect corresponding to that tolerability factor.

In some embodiments of the method according to the first aspect of the present disclosure, the plurality of tolerability factors comprises two or more medication side effects.

In some embodiments of the method according to the first aspect of the present disclosure, the two or more medication side effects comprises two or more of: weight gain, sexual dysfunction, daytime sedation, gastrointestinal distress, discontinuation reaction, affective flattening, metabolic syndrome, extrapyramidal side effects.

In some embodiments of the method according to the first aspect of the present disclosure, for each pharmacological treatment option, the respective tolerability rating is a weighted function of the respective ratings for the plurality of tolerability factors for that pharmacological treatment option, wherein respective weightings of the respective ratings for the plurality of tolerability factors are based on at least one of: patient-identified complaints or clinical concerns identified in the patient profile data; patient health history identified in the patient profile data; or one or more comorbidities identified in the diagnosis.

In some embodiments of the method according to the first aspect of the present disclosure, the indication of the personalized treatment plan in the electronic report comprises, for each pharmacological treatment option in the ordered listing of pharmacological treatment options, an indication of the respective tolerability rating for that pharmacological treatment option.

In some embodiments of the method according to the first aspect of the present disclosure, obtaining patient profile data for a particular patient comprises: obtaining preliminary patient profile data for the patient, the preliminary patient profile data for the patient comprising a preliminary diagnosis for the patient; determining whether the preliminary diagnosis for the patient is unsupported by the preliminary patient profile data; after determining that the preliminary diagnosis for the patient is unsupported by the preliminary patient profile data, generating a request for additional patient profile data for the patient, the additional patient profile data comprising a result of at least one additional evaluation test or interview of the patient; and obtaining the additional patient profile data for the patient in accordance with the request.

In some embodiments of the method according to the first aspect of the present disclosure, determining a diagnosis of the patient based on the patient profile data comprises determining the diagnosis of the patient based on the preliminary patient profile data for the patient and the additional patient profile data for the patient.

In some embodiments of the method according to the first aspect of the present disclosure, determining a diagnosis of the patient based on the patient profile data comprises: for a given diagnosis, determining one or more differential diagnoses to rule out; assessing the given diagnosis and the one or more differential diagnoses based on the patient profile data to identify a supported diagnosis; and identifying the supported diagnosis as the diagnosis of the patient.

In some embodiments of the method according to the first aspect of the present disclosure, determining the diagnosis of the patient based on the patient profile data further comprises: for the supported diagnosis, assessing one or more common comorbidities of the supported diagnosis based on the patient profile data to determine whether a given comorbidity is supported by the patient profile data; and for each comorbidity that is determined to be supported by the patient profile data, identifying the comorbidity in the diagnosis of the patient.

In some embodiments of the method according to the first aspect of the present disclosure, the health disorder comprises a mental health disorder.

In some embodiments of the method according to the first aspect of the present disclosure, the mental health disorder comprises major depressive disorder (MDD), general anxiety disorder (GAD), bipolar spectrum disorder (BP), post-traumatic stress disorder (PTSD), social anxiety disorder, panic disorder, obsessive-compulsive disorder (OCD), or insomnia.

In some embodiments of the method according to the first aspect of the present disclosure, the at least one evaluation test or interview includes at least one of: patient health questionnaire (PHQ-9), general anxiety disorder questionnaire (GAD-7), mood disorder questionnaire (MDQ), insomnia severity index (ISI), mental health quality of life questionnaire (MHQoL), or Sheehan disability scale (SDS).

In some embodiments of the method according to the first aspect of the present disclosure, the personalized treatment plan further comprises a non-pharmacological treatment component providing guidance regarding one or more non-pharmacological treatment interventions.

In some embodiments of the method according to the first aspect of the present disclosure, the one or more non-pharmacological treatment interventions comprises one or more adjunctive psychological interventions.

In some embodiments of the method according to the first aspect of the present disclosure, the one or more adjunctive psychological interventions comprise at least one of: Cognitive-Behavioral Therapy (CBT), Interpersonal Therapy (IT), Behavioral Activation (BA) and Mindfulness-Based Cognitive Therapy (MBCT).

In some embodiments of the method according to the first aspect of the present disclosure, the personalized treatment plan further comprises a neurostimulation treatment component providing guidance regarding whether neurostimulation treatment is recommended for the patient.

In some embodiments of the method according to the first aspect of the present disclosure, the personalized treatment plan further comprises a follow-up guidance component providing guidance for follow-up assessments of the patient's response to treatment of the health disorder.

In some embodiments of the method according to the first aspect of the present disclosure, the health disorder comprises a substance abuse disorder.

In some embodiments of the method according to the first aspect of the present disclosure, the health disorder comprises a pain management disorder.

In some embodiments of the method according to the first aspect of the present disclosure, the health disorder comprises a non-surgical medical speciality.

In some embodiments of the method according to the first aspect of the present disclosure, the non-surgical medical speciality comprises any one or more of: geriatric medicine, pain disorders, neurology, cardiology, rheumatology, endocrinology, and gastroenterology.

A second aspect of the present disclosure provides a device. The device comprises: one or more processors; memory; and one or more programs. The one or more programs are stored in the memory and configured to be executed by the one or more processors. The programs include instructions for obtaining patient profile data for a patient, the patient profile data comprising a result of at least one evaluation test or interview of the patient. The programs may also include instructions for determining, based on the patient profile data, a diagnosis of the patient. For example, the diagnosis may identify a health disorder of the patient. The programs may further include instructions for determining, based on the patient profile data and the diagnosis of the patient, a personalized treatment plan to address the health disorder of the patient. For example, the personalized treatment plan may include a pharmacotherapy component identifying an ordered listing of pharmacological treatment options, each pharmacological treatment option corresponding to a different pharmaceutical product for treatment of the health disorder. For example, ordering of the pharmacological treatment options in the ordered listing may be based on a ranking of the pharmacological treatment options for treatment of the health disorder based on the patient profile data and the diagnosis of the patient. The programs may further include instructions for generating an electronic report that includes an indication of the diagnosis of the patient, and an indication of the personalized treatment plan to address the health disorder of the patient.

A third aspect of the present disclosure provides a computer readable storage medium. The computer readable storage medium has stored therein instructions, which when executed by one or more processors, cause the one or more processors to perform operations for generating personalized diagnostic and treatment guidance. For example, the operations may include obtaining patient profile data for a patient, the patient profile data comprising a result of at least one evaluation test or interview of the patient. The operations may further include determining, based on the patient profile data, a diagnosis of the patient. For example, the diagnosis may identify a health disorder of the patient. The operations may further include determining, based on the patient profile data and the diagnosis of the patient, a personalized treatment plan to address the health disorder of the patient. For example, the personalized treatment plan may include a pharmacotherapy component identifying an ordered listing of pharmacological treatment options, each pharmacological treatment option corresponding to a different pharmaceutical product for treatment of the health disorder. Ordering of the pharmacological treatment options in the ordered listing may be based on a ranking of the pharmacological treatment options for treatment of the health disorder based on the patient profile data and the diagnosis of the patient. The operations may further include generating an electronic report that includes an indication of the diagnosis of the patient, and an indication of the personalized treatment plan to address the health disorder of the patient.

Other aspects and features of embodiments of the present disclosure will become apparent to those ordinarily skilled in the art upon review of the following description.

### BRIEF DESCRIPTION OF THE DRAWINGS

Reference will now be made, by way of example, to the accompanying drawings which show example embodiments of the present application, and in which:

FIG. 1 is a schematic diagram illustrating an example system architecture for generating diagnoses and treatment plans for patients according to some embodiments of the present disclosure;

FIG. 2 is a block diagram illustrating an example processing system suitable for implementing an electronic device in the system architecture of FIG. 1;

FIG. 3 is a block diagram illustrating an example processing system suitable for implementing another electronic device in the system architecture of FIG. 1;

FIG. 4 is a flowchart illustrating a method for generating personalized diagnostic and treatment guidance according to some embodiments of the present disclosure;

FIGS. 5A-5D are schematic diagrams depicting workflows for example use cases of the personalized diagnostic and treatment guidance provided in accordance with embodiments of the present disclosure;

FIG. 6 is a schematic diagram depicting various integration mechanisms to integrate the personalized diagnostic and treatment service with other services or systems in accordance with embodiments of the present disclosure;

FIGS. 7A-7I depicts portions of a user interface providing a guided screening process to collect patient profile data in accordance with embodiments of the present disclosure;

FIGS. 8A-8E depict examples of rule logic to arrive at treatment guidance for diagnoses involving major depressive disorder in accordance with embodiments of the present disclosure;

FIG. 9 depicts an example of rule logic to assess differential diagnoses and comorbidities for a given diagnosis in accordance with an embodiment of the present disclosure;

FIGS. 10A-10D depict example pages of an electronic report in accordance with an embodiment of the present disclosure;

FIGS. 11A-11F depict portions of a user interface providing another example of an electronic report that includes personalized diagnostic and treatment guidance in accordance with an embodiment of the present disclosure;

FIG. 12 depicts an example of a table of medication side effect gradings that may be used to determine tolerability ratings for the listed medications in accordance with an embodiment of the present disclosure;

FIGS. 13A and 13B depict an example of calculating a single tolerability rating for a medication as a function of multiple tolerability factors based on the side effect gradings of FIG. 12;

FIG. 14 depicts a table and a corresponding chart that show the respective single tolerability rating expressed as a rating out of five for nine of the medications listed in the table of FIG. 12 having the highest single tolerability ratings in accordance with an embodiment of the present disclosure;

FIG. 15 depicts four tables showing changes in the tolerability ratings of the medications for four different scenarios in which the tolerability factors related to weight gain, sexual dysfunction, daytime sedation and affective flattening, respectively, are more heavily weighted in the calculation of the tolerability rating in accordance with an embodiment of the present disclosure;

FIG. 16 illustrates an example of how an additional clinical benefits rating may be calculated for a medication in a manner similar to that used to calculate a tolerability rating for the medication in accordance with an embodiment of the present disclosure;

FIG. 17A depicts an example of a diagnostic causal graph in accordance with an embodiment of the present disclosure; and

FIG. 17B depicts an example of a treatment causal graph in accordance with an embodiment of the present disclosure.

Similar reference numerals may have been used in different figures to denote similar components.

In the drawings, embodiments are illustrated by way of example. It is to be expressly understood that the description and drawings are only for purposes of illustrating certain embodiments and are an aid for understanding. They are not intended to be a definition of the limits of the invention.

### DESCRIPTION OF EXAMPLE EMBODIMENTS

The present disclosure is made with reference to the accompanying drawings, in which certain non-limiting embodiments are shown. However, the description should not be construed as being limited to the embodiments set forth herein. Rather, these embodiments are provided as examples. Like numbers refer to like elements/components throughout. Separate boxes or illustrated separation of functional elements or modules of illustrated systems and devices does not necessarily require physical separation of such functions or modules, as communication between such elements can occur by way of messaging, function calls, shared memory space, and so on, without any such physical separation. As such, functions or modules need not be implemented in physically or logically separated platforms, although they may be illustrated separately for ease of explanation herein. Different devices can have different designs, such that while some devices implement some functions in fixed function hardware, other devices can implement such functions in a programmable processor with code obtained from a machine readable medium.

### Example System Architecture

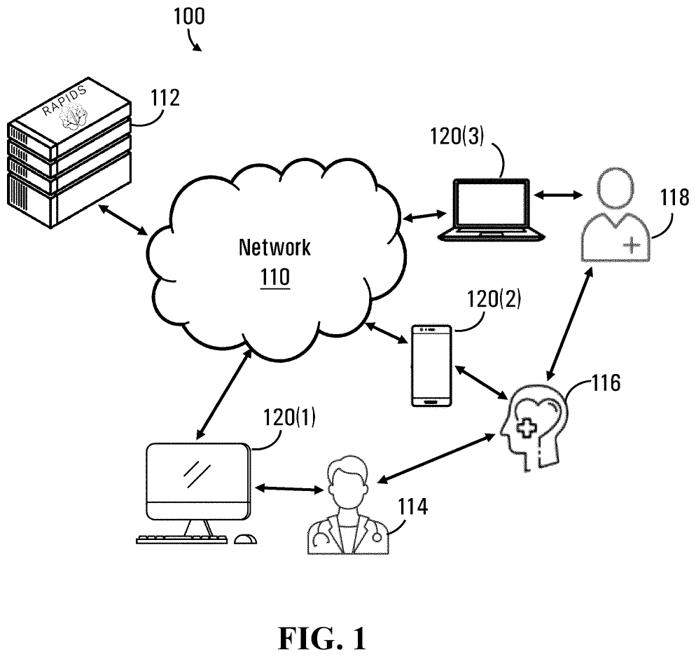

FIG. 1 is a schematic diagram illustrating an example system architecture 100 suitable for operating an electronic or virtual computing/serverless device 112 (herein referenced as an “electronic device”) that implements a computer-implemented method for personalized diagnostic and treatment guidance in accordance with a non-limiting example of the present disclosure. The system architecture 100 comprises a network 110, which can be a public data network such as the internet. The electronic device 112 is connected to the network 110, enabling the electronic device 112 to provide diagnostic and treatment guidance services that can be accessed through the network 110. In some cases, the diagnostic and treatment guidance services provided by the electronic device 112 may be referred to as Rapid Accurate Personalized Informed Diagnosis and Solutions (RAPIDS) services. As shown in FIG. 1, the system architecture further comprises three other electronic devices 120(1)-120(3) connected to the network 110, enabling the electronic devices 120(1)-120(3) to access one or more RAPIDS services provided by electronic device 112 through network 110. The system architecture 100 can include multiple different types of communication networks (not shown) in communication with the electronic devices 112 and 120(1)-120(3), and each of these communication networks can be connected directly or indirectly to the network 110.

In some applications, the electronic device 112 may be referred to as a server. In some applications, the electronic device 112 implements a virtual cloud server and the one or more services provided by the virtual cloud server are hosted and delivered over the network 110.

The electronic devices 120(1)-120(3) are collectively referred to as electronic devices 120 and in some applications, the electronic devices 120 may also be referred to as user devices because they include one or more user interfaces through which they can directly interface with a human user to receive and/or convey information. It should be appreciated that although the drawing shows three network-connected electronic devices 120, there is no particular limitation on the number of electronic devices.

The service(s) provided by the electronic device 112 may be reachable by the user devices 120 over the network 110 in various non-limiting ways. For example, user device 120(1) may establish a wired link with the network 110 in order to access services provided by the electronic device 112 over the network 110. On the other hand, user device 120(2) may access service(s) provided by the electronic device 112 by communicating with the network 110 via cellular communication links, and user device 120(3) may access service(s) provided by the electronic device 112 using a Wi-Fi wireless network which conforms to IEEE 802.11x standards (sometimes referred to as Wi-Fi®, although in other examples, other communication protocols may be used for the Wi-Fi wireless network).

User devices 120 may be any component (or collection of components) capable of interfacing with a user, e.g., carrying out a graphical user interface (GUI), and accessing services provided by electronic device 112. For example, in the embodiment of FIG. 1, user device 120(1) is a desktop console used by a user 114, user device 120(2) is a mobile phone used by a user 116 and user device 120(3) is a laptop used by a user 118. A graphical user interface (GUI) can be defined as functionality for displaying information for visualization by a user, receiving input from the user pertaining to the displayed information, associating the user input to the displayed information and taking an action based on the displayed information associated with the user input. In some cases, a GUI can be implemented by a combination of a display, a mouse/keyboard and an input/output controller, whereas in other cases, a GUI can be implemented by a touchscreen and touchscreen controller. Other embodiments are of course possible.

In various non-limiting embodiments, the user 116 may be a patient, the user 114 may be a healthcare professional such as the patient's physician or an independent medical examiner (IME), psychiatrist, or clinical assessor that is contracted by an insurance company to assess the patient, and the user 118 may be an interviewer that interacts with the patient to obtain additional information on an as needed basis as will be discussed in further detail later on. As shown in the embodiment of FIG. 1, the patient 116 may interact with the healthcare professional 114 and/or the interviewer 118 in person and/or through network 110. For example, the patient may interact with the healthcare professional 114 and/or the interviewer 118 through voice calls, video calls and/or written communications such as emails or text messages.

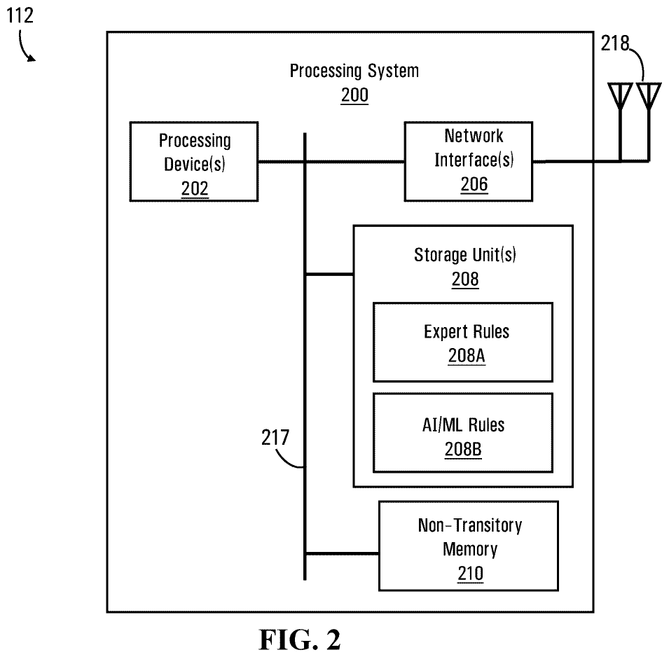

Configurations of the electronic device 112 will be discussed in greater detail below with reference to the simplified block diagram of FIG. 2. The electronic device 112 comprises a processing system 200. The example processing system 200 described below, or variations thereof, may be used to implement certain functionality of the electronic device 112. However, other processing system architectures may be suitable for implementing the electronic device 112 and may include components different from those discussed below. Although FIG. 2 shows a single instance of each component in the processing system 200, there may be multiple instances of each component.

The processing system 200 may include one or more processing devices 202, such as a central processing unit (CPU), a graphics processing unit (GPU), a tensor processing unit (TPU), a neural processing unit (NPU), an application-specific integrated circuit (ASIC), a field-programmable gate array (FPGA), a dedicated logic circuitry, or combinations thereof.

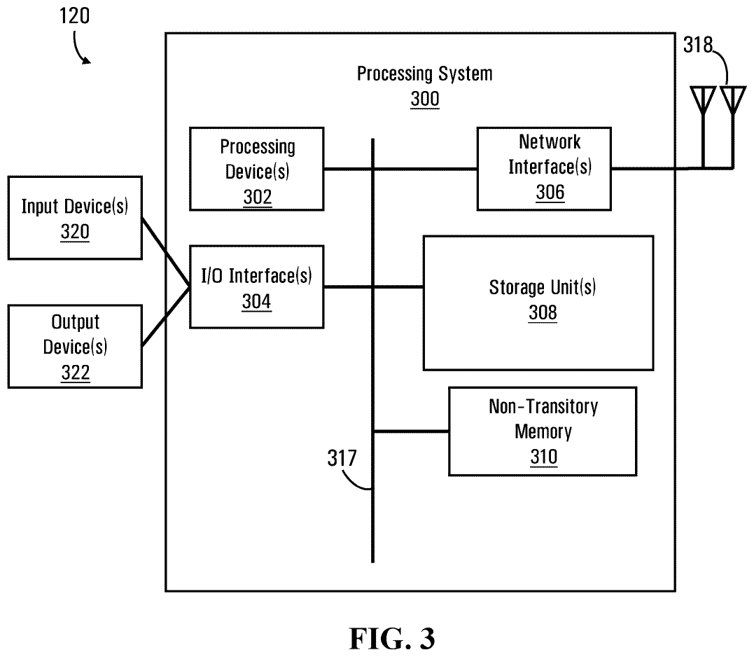

The processing system 200 may further include one or more network interfaces 206 for wired or wireless communication with the communication network 110 or peer-to-peer communication with other processing systems, such as a processing system 300 (see FIG. 3) of a user device 120. The network interface(s) 206 may include wired links (e.g., Ethernet cable) and/or wireless links (e.g., one or more antennas) for intra-network and/or inter-network communications. The network interface 206 may be connected to one or more antennas 218 of the electronic device, which are configured to facilitate wireless communication that may be implemented by the network interface 206.

The processing system 200 may also include or have access to one or more storage units 208, which may include a mass storage unit such as a solid state drive, a hard disk drive, a magnetic disk drive and/or an optical disk drive. In some examples, the storage unit(s) 208 may implement an expert rules database 208A that stores information pertaining to rules logic for guiding diagnostic processes, determining diagnoses and generating treatment plans. In some examples, the storage unit(s) 208 may also or instead implement an artificial intelligence or machine learning (AI/ML) rules database 208B that stores AI/ML model(s) trained to provide probabilistic diagnostic and treatment insights. For example, the insights provided by the AI/ML model(s) may be used to augment or refine the deterministic rules logic implemented by a rules engine, e.g., a rules engine configured to carry out the rules logic indicated by the information stored in the expert rules database 208A. The usefulness of the expert rules database 208A and the AI/ML rules database 208B will become apparent later on from a reading of this document.

The processing system 200 may include one or more non-transitory memories 210, which may include a volatile or non-volatile memory (e.g., a flash memory, a random-access memory (RAM), and/or a read-only memory (ROM)). The non-transitory memory(ies) 210 may store instructions for execution by the processing device(s) 202, such as to carry out example methods described in the present disclosure. The memory(ies) 210 may store other software (e.g., instructions for execution by the processing device(s) 202), such as an operating system and other applications/functions. In some examples, one or more data sets and/or modules may be provided by an external memory (e.g., an external drive in wired or wireless communication with the processing system 200) or may be provided by a transitory or non-transitory computer-readable medium. Examples of non-transitory computer readable media include a RAM, a ROM, an erasable programmable ROM (EPROM), an electrically erasable programmable ROM (EEPROM), a flash memory, a CD-ROM, or other portable memory storage.

There may be a bus 217 providing communication among components of the processing system 200, including the processing device(s) 202, network interface(s) 206, storage unit(s) 208, and/or memory(ies) 210. The bus 217 may be any suitable bus architecture including, for example, a memory bus, a peripheral bus or a video bus.

FIG. 3 is a block diagram of an example simplified processing system 300, which may be used to implement any or each of the user devices 120. Components of the processing system 300 are similar to those of the processing system 200 as shown in FIG. 2. A notable exception is the addition of one or more input/output (I/O) controllers 304, to enable interfacing with one or more input devices 320 and/or output devices 322. That is to say, the electronic device 112 of FIG. 2 might not include any display (including touchscreen), mouse, keyboard or microphone, as it might not be designed for interfacing with a user. Rather, the main function of the electronic device 112 includes delivering the RAPIDS service through user devices 120 over network 110.

In particular, the processing system 300 may include one or more network interfaces 306 for wired or wireless communication with the network 110 or peer-to-peer communication with the processing system 200 of the electronic device 112, depending on the implementation.

The processing system 300 may include one or more processing devices 302, such as a central processing unit (CPU), a graphics processing unit (GPU), a tensor processing unit (TPU), a neural processing unit (NPU), an application-specific integrated circuit (ASIC), a field-programmable gate array (FPGA), a dedicated logic circuitry, or combinations thereof.

There may be a bus 317 providing communication among components of the processing system 300, including the processing device(s) 302, I/O interface(s) 304, network interface(s) 306, storage unit(s) 308, and/or memory(ies) 310. The bus 317 may be any suitable bus architecture including, for example, a memory bus, a peripheral bus or a video bus.

In FIG. 3, an input device(s) 320 and an output device(s) 322 are shown as external to the processing system 200. The input device(s) 320 may include at least one of a keyboard, a mouse, and a microphone, which receive input from a user. The output device(s) 322 may include at least one of a display and a loudspeaker, which provide audio and visual output to a user. In other examples, one or more of the input device(s) 320 and/or the output device(s) 322 may be integrated together and/or with the processing system 300. For example, the input device 320 and the output device 322 may be integrated as a single component, such as a touch-sensitive display screen which, together with the I/O interface 304 being implemented as a touchscreen controller, carries out a graphical user interface. In that case, a user may interact with the GUI primarily through finger contacts and gestures on the touch-sensitive display.

### Method for Personalized Diagnostic and Treatment Guidance

Referring again to FIG. 2, one of the methods carried out by the processing device(s) 202 executing the computer-readable instructions in the memory(ies) 210 includes obtaining patient profile data for a patient (e.g., patient profile data for the patient 116 submitted by the healthcare professional 114 via user device 120(1), by the trained interviewer 118 via user device 120(3) and/or by the patient 116 themself via user device 120(2), determining a diagnosis for the patient based on the patient profile data, determining a personalized treatment plan based on the patient profile data and the diagnosis and generating an electronic report that provides the diagnostic assessment and personalized treatment plan for the patient.

As a result of this method, an electronic report that includes personalized diagnostic and treatment guidance for a patient (e.g., the patient 116) is generated for display on the graphical user interface of one or more user devices (e.g., one or more of the user devices 120).

The personalized treatment plan includes a pharmacotherapy component identifying an ordered listing of pharmacological treatment options, each pharmacological treatment option corresponding to a different pharmaceutical product for treatment of a health disorder identified in the diagnosis. For example, each pharmacological treatment option may correspond to a different pharmaceutical product that is either approved or commonly used off-label by medical specialists for treatment of a health disorder identified in the diagnosis. Ordering of the pharmacological treatment options in the ordered listing may be based on a ranking of the pharmacological treatment options for treatment of the health disorder based on the patient profile data and the diagnosis of the patient. This helps a user, such as the healthcare professional 114 or the patient 116, identify the preferred pharmacological treatment options from among the multitude of treatment options that may be approved or commonly used off-label by medical specialists for treatment of a given disorder.

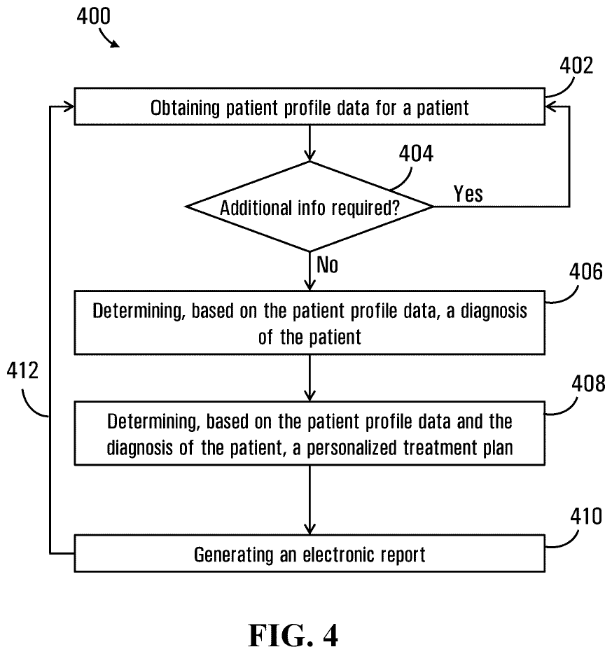

With reference therefore to FIG. 4, an example method 400 may be implemented by the electronic device 112 as discussed above.

### Step 402:

At step 402, patient profile data is obtained for a patient, the patient profile data comprising a result of at least one evaluation test or interview of the patient. For example, in some cases obtaining the patient profile data at step 402 may involve receiving patient profile data for the patient 116 submitted by the user 114 at user device 120(1). As discussed above and in further detail below, in some cases user 114 may be the patient's family doctor (e.g., a general practitioner without specific training or expertise related to the diagnosis or treatment of one or more health disorders the patient may be experiencing) or another healthcare profession, such as nurse or a trained interviewer that has been contracted to assess the patient (e.g., as part of an insurance claim assessment process).

In some implementations, user 114 or user 118 may be guided through a screening process to collect at least part of the patient profile data using validated diagnostic tools to ensure that diagnosis of a given health condition can be made on the basis of the diagnostic test results that have been validated as being the most relevant to diagnosing that particular health condition. For example, in the case of a patient exhibiting symptoms of a mental health disorder such as MDD or generalized anxiety disorder (GAD), the RAPIDS service provided by electronic device 112 may guide a user, such as user 114 via user device 120(1) or user 118 via user device 120(3), to collect additional patient data such as administering at least a minimum set of additional diagnostic screening such as the Patient Health Questionnaire-9 (PHQ-9), which is a multipurpose instrument for screening, diagnosing, monitoring and measuring the severity of depression, and the General Anxiety Disorder-7 questionnaire (GAD-7), which is a seven-item instrument that is used to measure or assess the severity of generalized anxiety. In some cases, further test/questionnaires may also be suggested, such as, Mood Disorder Questionnaire (MDQ), which is a checklist that serves as an effective instrument for screening patients who have a history of a manic episode associated with bipolar disorder, Insomnia Severity Index (ISI), which is a seven-item self-report questionnaire assessing the nature, severity, and impact of insomnia, Mental Health Quality of Life Questionnaire (MHQoL), which is a standardized, self-administered measure of quality of life that has been developed for use in people with mental health problems, or Sheehan Disability Scale (SDS), which is a brief, patient-rated measure of disability and impairment.

### Step 404:

At step 404, a determination is made as to whether additional information is required in order to validate a preliminary diagnosis (e.g., a preliminary diagnosis that may have been included in the patient profile data obtained at step 402) and/or in order to generate a new diagnosis. If no additional information is required, the method 400 proceeds to step 406 (the “No” path in FIG. 4). On the other hand, if electronic device 112 determines that additional information is required (e.g., determined using the expert rules logic stored in the expert rules database 208a), the method 400 returns to step 402 (the “Yes” path in FIG. 4), in which one or more of the users 114 and 118 may be prompted to obtain and provide the additional information by administering one or more additional tests or evaluations to the patient 116. For example, if the PHQ9 confirms depression, then the RAPIDS system rules may trigger a request for completion of an MDQ assessment scale to assess for bipolar. In this way, the system guides the healthcare professional interviewing the patient as to which standardized assessment scale(s) is/are required to ensure a comprehensive assessment without requiring the patient to complete unnecessary scales. In some cases, electronic device 112 may prompt the patient 116 to provide the additional information (e.g., if the additional information can be obtained by a self-administered test or evaluation or answering additional health related questions).

### Step 406:

At step 406, the electronic device 112 determines a diagnosis of the patient based on the patient profile data. For example, the diagnosis may be determined using the expert rules logic stored in the expert rules database 208a. In some implementations, determining the diagnosis of the patient based on the patient profile data involves ruling out one or more potential differential diagnoses associated with a candidate diagnosis and/or assessing the patient for one or more comorbidities commonly present with a health disorder included in the candidate diagnosis. A given diagnosis identifying a given health disorder may have one or more associated differential diagnoses corresponding to health disorders that may present with some clinical features similar to that of the given health disorder. For example, as noted above, in the context of mental health, for a diagnosis of MDD, the MDQ assessment scale may be used to rule out (or rule in) bipolar disorder as a differential diagnosis for the MDD diagnosis. Similarly, the patient may also be assessed for anxiety and insomnia. The electronic device 112 then determines a supported diagnosis from the diagnostic assessment of the patient based on the patient profile data and the analysis above. This can be a confirmation of the given diagnosis or a differential diagnosis supported by the evidence.

### Step 408:

At step 408, the electronic device 112 determines, based on the patient profile data and the diagnosis of the patient, a personalized treatment plan to address a health disorder identified in the diagnosis. For example, the personalized treatment plan may be determined using the expert rules logic stored in the expert rules database 208a. The personalized treatment plan comprises a pharmacotherapy component identifying an ordered listing of pharmacological treatment options, each pharmacological treatment option corresponding to a different pharmaceutical product approved or commonly used off-label by medical specialists for treatment of the health disorder. As discussed above, the ordering of the pharmacological treatment options in the ordered listing may be based on a ranking of the pharmacological treatment options for treatment of the health disorder based on the patient profile data and the diagnosis of the patient. For example, if the patient profile data indicates that the patient is currently on an antidepressant (ADT) that is not well tolerated by the patient, the personalized treatment plan generated by electronic device 112 may suggest switching the patient to another preferred ADT with a different mechanism of action (e.g., switching from a selective serotonin reuptake inhibitor (SSRI) such as Fluoxetine/Prozac to a Norepinephrine and Dopamine Reuptake Inhibitor (NDRI) such as Bupropion XL/Wellbutrin XL).

In some implementations, the ordered listing of pharmacological treatment options includes only a partial subset of the highest ranked pharmacological treatment options corresponding to pharmaceutical products approved or commonly used off-label by medical specialists for treatment of the health disorder. This can simplify the selection of an appropriate medication by reducing the recommended treatment choices from a long list of clinically recognized treatment options.

In some cases, the pharmacotherapy component of the personalized treatment plan for the patient includes pharmacological treatment guidance providing at least one of the following: guidance for starting a pharmacological treatment option from among the ordered listing of pharmacological treatment options; guidance for stopping a current pharmacological treatment option identified in the patient profile data; guidance for optimizing a dosage of a current pharmacological treatment option identified in the patient profile data; guidance for switching from the current pharmacological treatment option to a different pharmacological treatment option from among the ordered listing of pharmacological treatment options; and guidance for augmenting the current pharmacological treatment option with another different pharmacological treatment option from among the ordered listing of pharmacological treatment options.

In some cases, the diagnosis determined by electronic device 112 may identify multiple health disorders of the patient. For example, the electronic device 112 may identify MDD as a primary health disorder of the patient 116 based on the PHQ-9 score in the patient profile data, and may also identify insomnia as a second comorbid health disorder of the patient 116 based on the result of an ISI score in the patient profile data. In such cases, the personalized treatment plan may address treatment of the primary health disorder and the second comorbid disorder of the patient. For example, in such cases the pharmacotherapy component of the personalized treatment plan may include a first ordered listing of pharmacological treatment options for treatment of the primary health disorder and a second ordered listing of pharmacological treatment options for treatment of the second comorbid health disorder. Similar to the ordering of the pharmacological treatment options in the first ordered listing for treatment of the primary health disorder, the ordering of the pharmacological treatment options in the second ordered listing for treatment of the second comorbid health disorder may be based on a ranking of the pharmacological treatment options for treatment of the second comorbid health disorder based on the patient profile data and the diagnosis of the patient. In some cases, the ordering of the pharmacological treatment options for treatment of the primary health disorder in the first ordered listing may consider both treatment of the primary health disorder and treatment of the second comorbid disorder. For example, in the case of a diagnosis of MDD with comorbid insomnia, the ordered listing of pharmacological treatment options for treatment of MDD may exclude treatments that potentially cause insomnia or other sleep related side effects.

As noted above, in some cases the diagnosis determined by electronic device 112 may identify multiple health disorders of the patient. In such cases, one of the health disorders may be identified as the primary health disorder of the patient based on a hierarchy for treatment of health disorders. The remaining health disorders may be identified as comorbidities of the primary health disorder, also in accordance with the hierarchy for treatment. For example, in the context of mental health, for a diagnosis that includes bipolar disorder, GAD and insomnia, bipolar would be identified as the primary health disorder with GAD identified as a second comorbid health disorder and then insomnia as a third comorbid health disorder. In general, for a patient with multiple comorbid conditions, the comorbid disorders should be treated according to a priority based on the severity of the disorder and the risk of harm to the patient or others. For that reason, in some embodiments, the personalized treatment plan addresses treatment of the primary health disorder and treatment of the health disorders identified as comorbidities to the primary health disorder in accordance with a priority based on the hierarchy for treatment of health disorders. In other words, the treatment guidance provided in the report may suggest that the most severe/risky conditions be treated first. For example, if the patient has bipolar mania, GAD, and ADHD, the mania typically needs to be addressed urgently and the GAD and ADHD may be addressed once the mania has been addressed.

In some embodiments, the ranking of the pharmacological treatment options is further based on a respective tolerability rating for each pharmacological treatment option. In some implementations, the respective tolerability rating for each pharmacological treatment option may be a single rating that is a function of respective ratings for a plurality of tolerability factors for that pharmacological treatment option. For example, the plurality of tolerability factors for a given pharmacological treatment option may correspond to potential side effects from that pharmacological treatment option, wherein the respective rating for a given tolerability factor indicates a likelihood of experiencing the side effect corresponding to that tolerability factor. For example, in the context of antidepressants for the treatment of MDD, the tolerability factors may include side effects such as weight gain, sexual dysfunction, daytime sedation, gastrointestinal distress, discontinuation reaction or affective flattening.

In some embodiments, the tolerability rating may be personalized to the patient through the use of personalized weighting of the tolerability factors based on patient-specific data. For example, in some embodiments the respective tolerability rating for each pharmacological treatment option is a weighted function of the respective ratings for the plurality of tolerability factors for that pharmacological treatment option, wherein respective weightings of the respective ratings for the plurality of tolerability factors are based on at least one of: patient-identified complaints or clinical concerns identified in the patient profile data; patient health history identified in the patient profile data; or one or more comorbidities identified in the diagnosis.

In view of the above, it should be appreciated that in some embodiments the RAPIDS recommended treatment plan is arrived at by assessing whether the patient should be on medication based on their current diagnosis and severity, and, if so, the pharmacological treatment rules simplify the selection of an appropriate medication by ranking and/or reducing the recommended treatment choices from a long list of medications (often 20 or 30) approved by FDA/Health Canada/other national drug approval bodies or commonly used off-label by medical specialists to treat a diagnosed mental health condition. For example, the recommended medication choices may be limited to only the top three highest ranked medication choices. These top three medication choices may be selected based on the latest evidence regarding the effectiveness for that diagnosis (or co-morbid diagnosis), the medication tolerability, and medication side effects personalized to the patient. In some embodiments, if the patient is currently prescribed medication, RAPIDS assesses if it is the correct medication, based on the diagnosis, and whether it is effective and well tolerated, using patient and clinician feedback and validated clinical scales. Additionally, dose optimization may be ascertained for that medication, e.g., based on the FDA/Health Canada approved dose ranges as well as dose ranges recognized by medical specialists. Based on the above, the system may recommend maintaining the current medication and dose, optimizing the dose, adding a second medication, changing the medication or stopping the medication as indicated above. Non-pharmacological treatment guidance may be similarly arrived at by having the system select appropriate treatment options for that condition based on recognized standards and evidence based practice. These may include treatment options such as neurostimulation, cognitive behavioral therapy, interpersonal therapy, physical activity, mindfulness meditation as well as evidence-based natural health options such as melatonin to assist with sleep.

### Step 410:

At step 410, the electronic device 112 generates an electronic report that includes the personalized diagnostic and treatment guidance for the patient (e.g., the patient 116) for display on the graphical user interface of one or more user devices (e.g., one or more of the user devices 120). For example, in the context of an insurance claim assessment for an employee disability claim, the electronic report generated at step 410 may be made accessible to an insurance industry case manager, which may in turn choose to
provide the report to the employee/claimant's primary care provider, who may use the guidance to improve their patient's path to recovery; and/or
recommend to the employer that the employee be placed on a return to work intervention program, where the RAPIDS report may be used to provide a more accurate reflection of the patient's current clinical needs.

In such a scenario, because the feedback the RAPIDS service provides is based on established clinical guidelines built on empirical evidence, it should support the employee to return to work sooner and lower the risk of relapse or progression to long-term disability.

### Example Use Cases

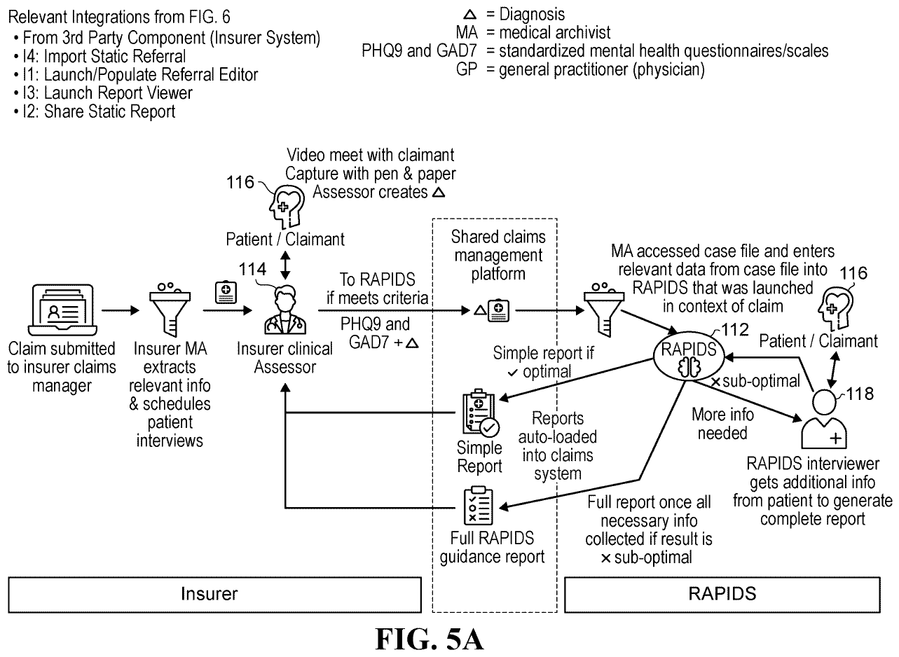

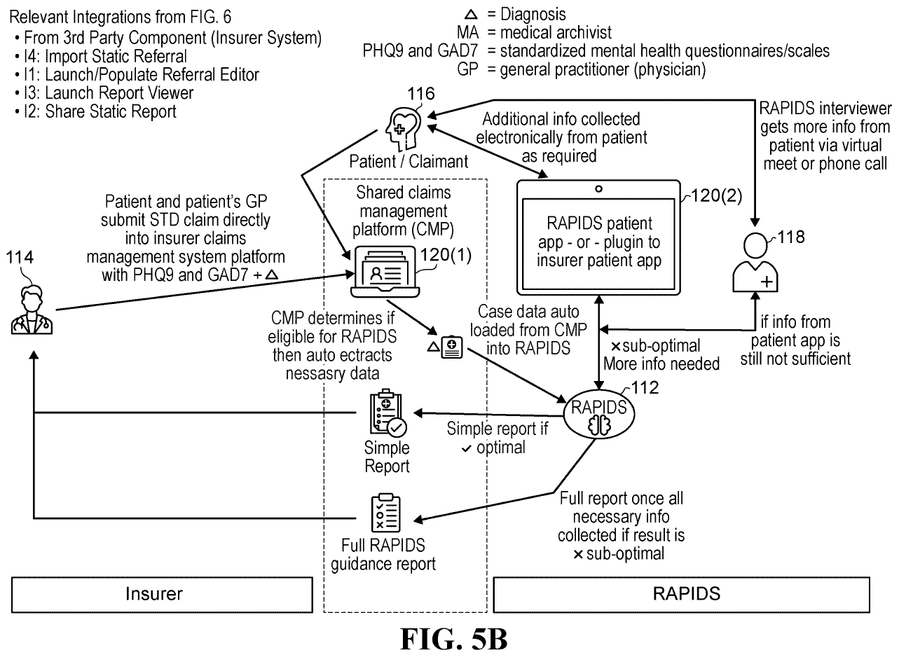

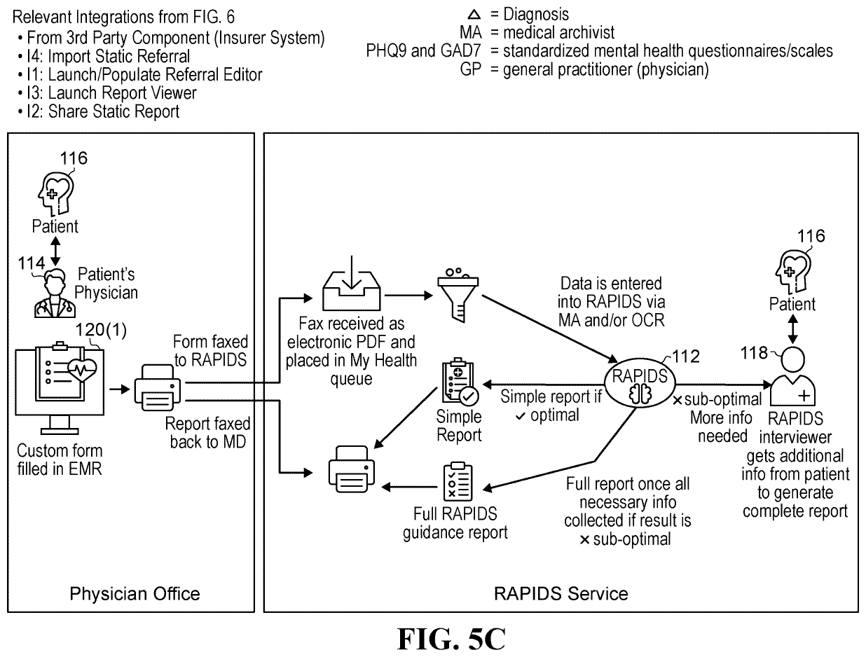

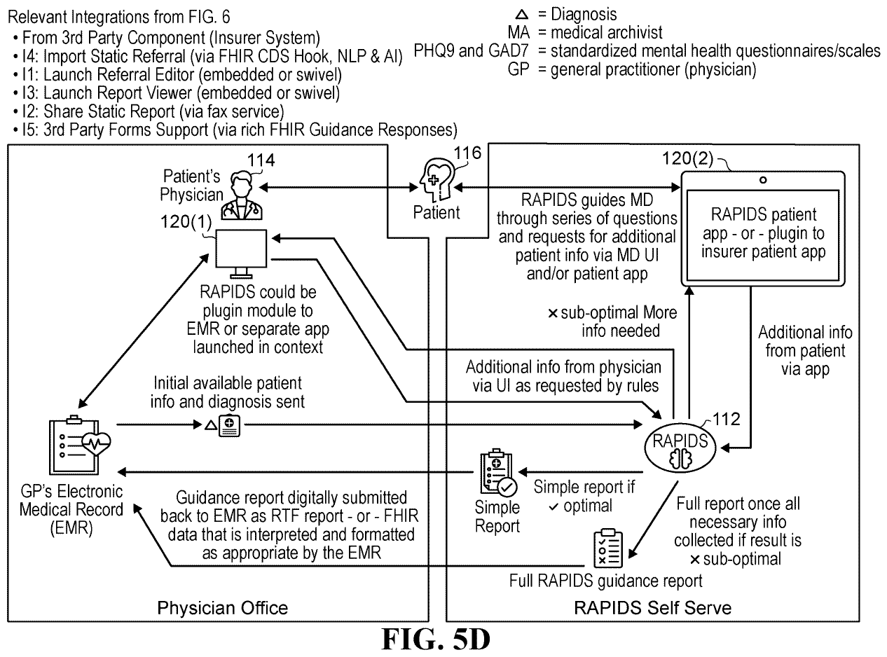

FIGS. 5A-5D are schematic diagrams depicting workflows for example use cases of the personalized diagnostic and treatment guidance provided by the RAPIDS service in accordance with embodiments of the present disclosure.

FIG. 5A is a schematic diagram depicting a workflow for an example use case of the personalized diagnostic and treatment guidance provided by the RAPIDS service for mental health disability claim assessments.

As shown in FIG. 5A, when a mental health disability claim is received by the insurance claims coordinator, they will assess if the claim meets the eligibility criteria for the RAPIDS service. Criteria for eligibility to use the RAPIDS service for a disability claim are based on the coverage of rules and content in the RAPIDS system for the patient's mental health condition. For example, in one non-limiting example embodiment, the acceptance criteria could include the following:
The working diagnosis submitted to the insurer in the employee disability claim includes one of the following covered conditions: Major depressive disorder (MDD); Generalized anxiety disorder (GAD); Bipolar disorder I or II (BPI, BPII); Insomnia.
The assessment scale results are positive for PHQ-9 (depression) or GAD-7 (anxiety) with a score >=5 (Mild and up)
The patient is of working age as of the date of referral: at least 18 years old and under 65.
The patient is able to participate in the RAPIDS interview. (i.e. sufficiently healthy, both mentally and physically, to engage in a conversation).

In some implementations, the acceptance criteria may further screen for clear secondary mental health comorbidities. For example, the acceptance criteria may screen out a patient having a clear secondary mental health comorbidity that is outside the scope that can be handled by a current rule set. For example, secondary mental health comorbidities such as ADHD and/or PTSD may be outside the scope of some rule sets. In other implementations, the acceptance criteria may not screen for secondary mental health comorbidities. In other words, in some cases even patients having a clear secondary mental health comorbidity may be accepted.

It should be appreciated that the foregoing example criteria are provided as non-limiting illustrative examples only. More generally, the applicable acceptance criteria are implementation-specific.

If the claim meets the criteria, the claim's coordinator will submit it, along with the patient's medical record, to the RAPIDS service via the insurer's claims management system (CMS), a secure and integrated platform.

A RAPIDS medical archivist (MA) will access the claim file and medical record from the RAPIDS queue in the insurer CMS. A medical archivist is a licensed professional responsible for managing and coding medical records and maintaining standards of privacy and disclosure for them in accordance with laws, regulations and policies.

In some embodiments, the MA may then launch the RAPIDS User Interface (UI). In some embodiments, only the Patient ID and Claim ID will be securely passed from the insurance CMS to the RAPIDS system, so that the RAPIDS guidance report can be accurately matched back to the correct insurance case. In some embodiments, secure integration with the insurer's claims system allows digitally passing the subset of data to initiate the first set of rules in the RAPIDS rules engine. In some embodiments, there may be no integration of the insurance CMS and the RAPIDS system. In such cases, the patient identifiers may be input manually from the patient file submitted into the CMS.

The MA will then read the patient record in order to find and manually enter the initial patient data that was not digitally passed from the CMS required by the RAPIDS engine including any commonly used assessment scales completed by physician 114 such as the PHQ-9 and GAD-7.

The RAPIDS rules will then trigger the UI to request any additional patient information, if required, as discussed earlier.

After all required information from the patient's medical file has been entered into RAPIDS, the system will return a response of either:
a. Optimal—the working diagnosis is validated, and the treatment plan is deemed optimal. At this point, a brief confirmatory report will be issued back to the insurance company via their CMS.
b. Suboptimal: the diagnosis is not validated and/or the treatment plan is sub-optimal or the system has insufficient information to complete the guidance

Any additional information required would be collected by a trained RAPIDS healthcare professional or trained clinical interviewer 118 who schedules a remote interview with the patient 116 to ask additional questions as prompted by the RAPIDS system. The RAPIDS healthcare professional 118 may request additional standardized mental health assessment screening scales that were not supplied in the initial submission (such as the PHQ-9, GAD-7, MDQ or ISI) be completed by the claimant via the secure CMS′ patient portal.

Once all the newly collected information is entered into the RAPIDS decision engine, a full assessment guidance report will be created by the system indicating:
a. If the working diagnosis is confirmed. If not, what the suggested diagnosis is with a clear indication of the basis for the suggested diagnosis (e.g. assessment scales scores, . . . ).
b. Guidance regarding an optimal treatment plan (with different modalities), again with clear background information supporting the guidance (e.g.: patient's concern with weight gain, sexual dysfunction . . . etc.).
c. Additional reference and educational material, including detailed drug information cards for each of the medication options. Education is a key aspect of the RAPIDS service.

FIG. 5B is a schematic diagram depicting a workflow for another example use case of the personalized diagnostic and treatment guidance provided by the RAPIDS service for mental health disability claim assessments. The example of FIG. 5B differs from the example of FIG. 5A in that in the example of FIG. 5A the RAPIDS system and the shared claims management system were essentially separate with minimal integration, whereas in the example of FIG. 5B the two systems are more integrated. As a consequence, in the example of FIG. 5B, the patient 116 and/or the patient's physician 114 can submit the claim directly into the insurer's claim management platform (CMP), and the CMP can determine if the claim is eligible for the RAPIDS service and, if so, auto extract the necessary data. The extracted case data may then be auto loaded from the CMP into the RAPIDS engine for analysis. The remainder of the workflow depicted in FIG. 5B generally follows that of the workflow depicted in FIG. 5A. One notable difference is that if additional information is required from the patient, it may be collected electronically from the patient through a RAPIDS patient app or through a plugin to the insurer's patient app on the patient's user device 120(2).

FIG. 5C is a schematic diagram depicting a workflow for an example use case of the personalized diagnostic and treatment guidance provided by the RAPIDS service for primary patient care. The initial stages of this workflow generally mirror the conventional process used today by GPs to request a consult from a medical specialist. However, as discussed previously, the wait time for a psychiatrist is often many months, whereas the delivery of the guidance report for a RAPIDS system analysis is just a few days or even as quickly as real time when used directly by physicians in the primary care setting.

The workflow begins with the physical filling in a custom form in an electronic medical record (EMR) system. The filled form with the patient profile data is faxed to the RAPIDS service, which receives the fax as an electronic PDF that is then placed in a processing queue.

Data from the received fax is then entered into RAPIDS by a RAPIDS MA and/or auto-extracted via optical character recognition (OCR) processing or an intelligent data extraction software process.

The remainder of the workflow depicted in FIG. 5C generally follows that of the workflow depicted in FIG. 5A. One notable difference is that in FIG. 5C the report generated by the RAPIDS service is then faxed back to the physician's office.

FIG. 5D is a schematic diagram depicting a workflow for another example use case of the personalized diagnostic and treatment guidance provided by the RAPIDS service for primary patient care. The example of FIG. 5D differs from the example of FIG. 5C in that in the example of FIG. 5C the RAPIDS system and the physician's EMR system were essentially separate with minimal integration, whereas in the example of FIG. 5D the two systems are more integrated. For example, the RAPIDS service may be integrated as a plugin module to the EMR system or as a separate application that is launched in context on the physician's user device 120(1). As a consequence, in the example of FIG. 5D, when the physician 114 enters a suspected mental health diagnosis for the patient 116 into the EMR system, the EMR system may suggest using RAPIDS to validate the diagnosis. If authorized to do so, the EMR system may then submit the patient profile data, including the initial available patient information and preliminary diagnosis by physician 114, to the RAPIDS service. The RAPIDS service may then guide the physician 114 and/or the patient 116 through a series of questions and requests for additional patient information via a user interface on the physician's user device 120(1) and/or via the user interface of a patient app on the patient's user device 120(2), as requested by the rule logic. The remainder of the workflow depicted in FIG. 5D generally follows that of the workflow depicted in FIG. 5C. One notable difference is that the guidance report generated by the RAPIDS service in FIG. 5D is digitally submitted back to the EMR, e.g., as a rich text format (RTF) report or, in some cases it may be provided in the form of Fast Health Interoperability Resources (FHIR) data that is interpreted and formatted as appropriate by the EMR system. FHIR is a set of standards to format and exchange health data.

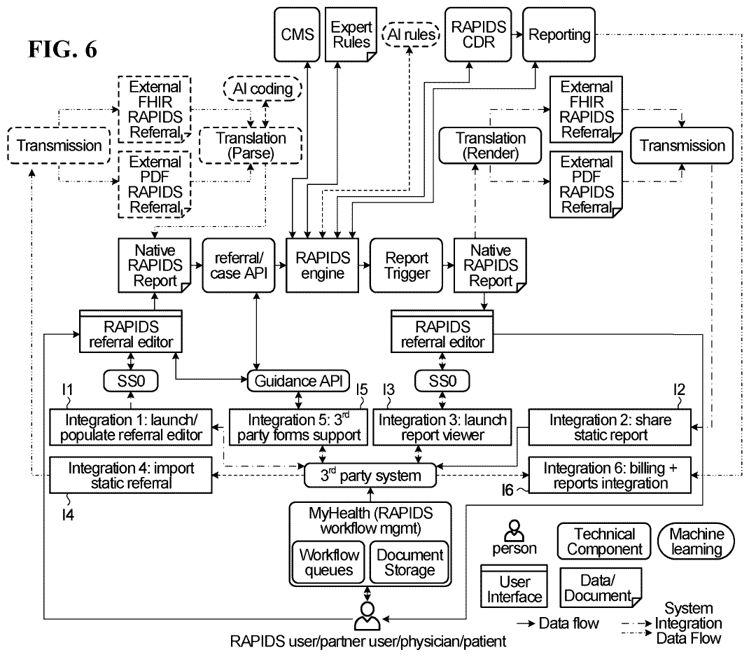

FIG. 6 is a schematic diagram depicting various integration mechanisms to integrate the personalized diagnostic and treatment service of RAPIDS with other services or systems, such as the claims management platform of FIGS. 5A and 5B and/or the EMR system of FIGS. 5C and 5D, in accordance with embodiments of the present disclosure.

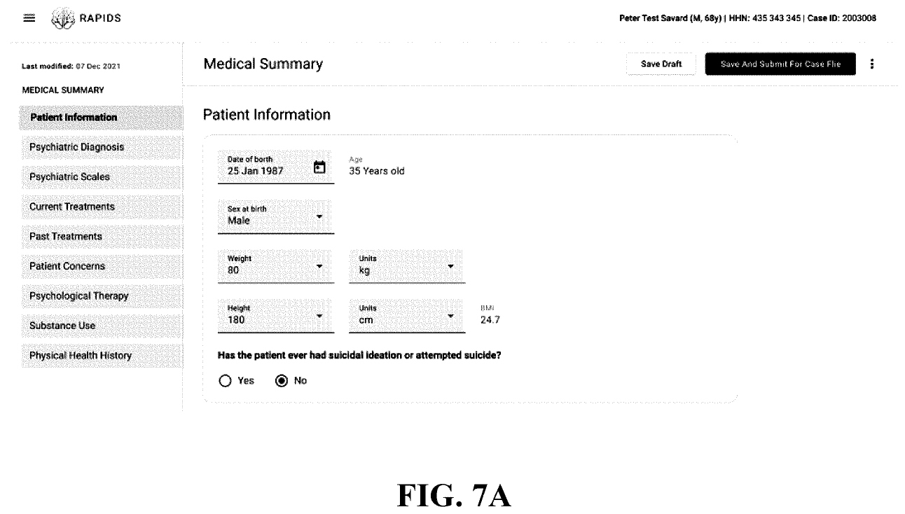

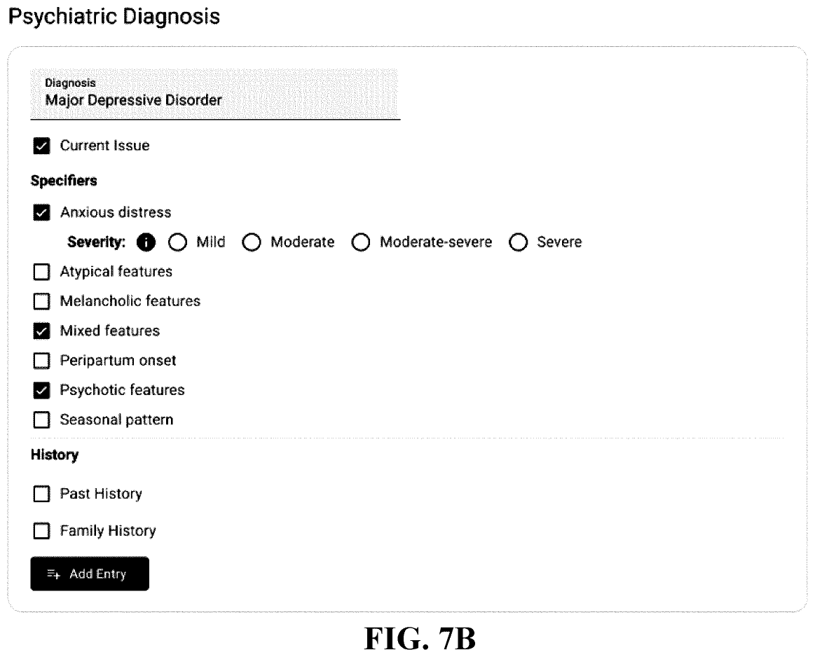

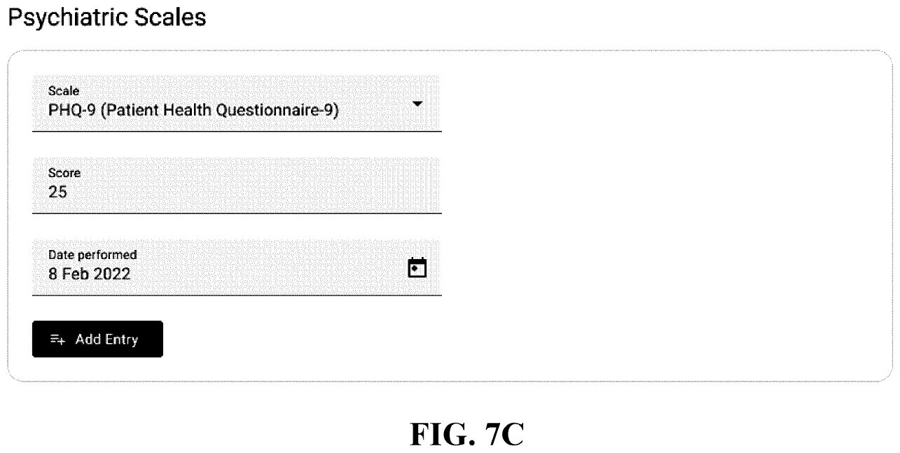

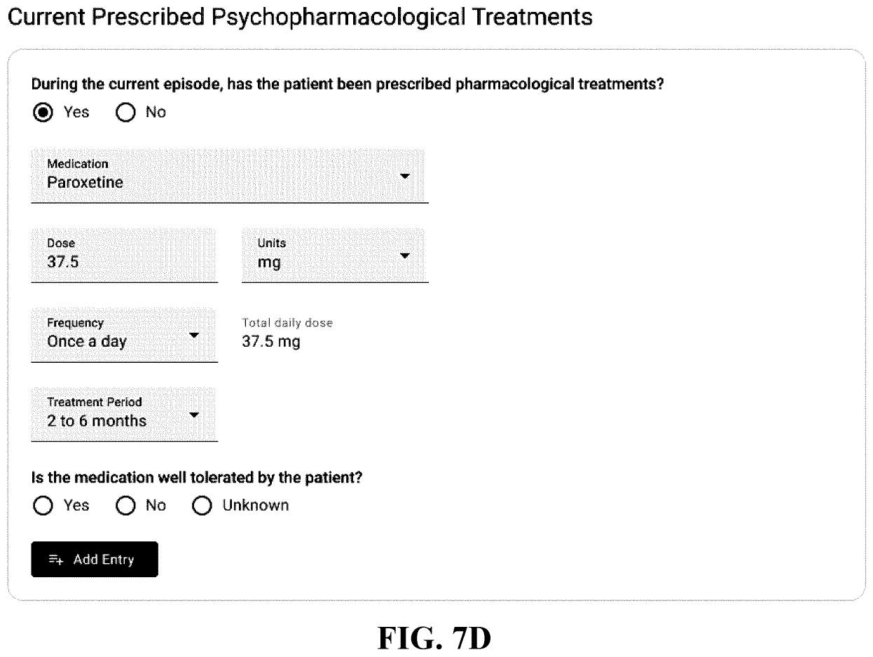

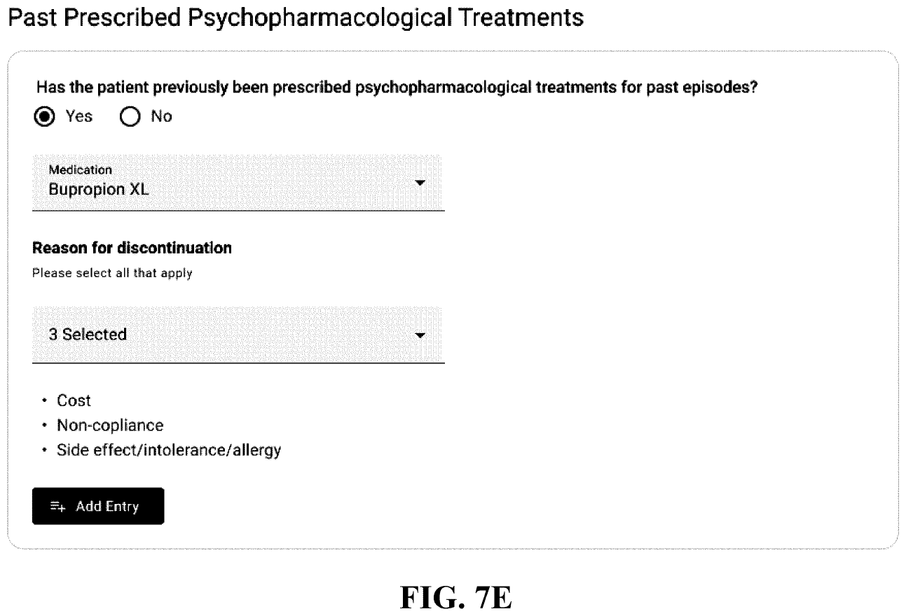

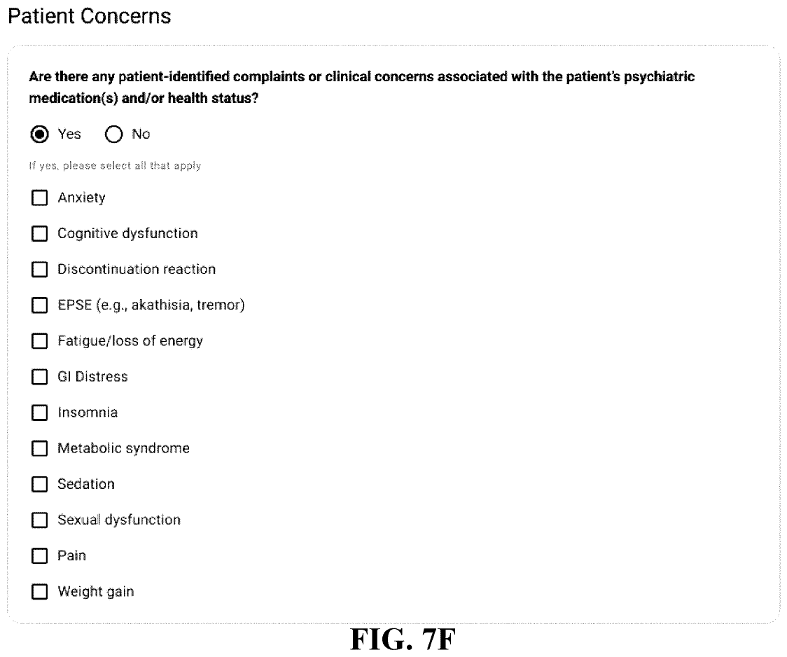

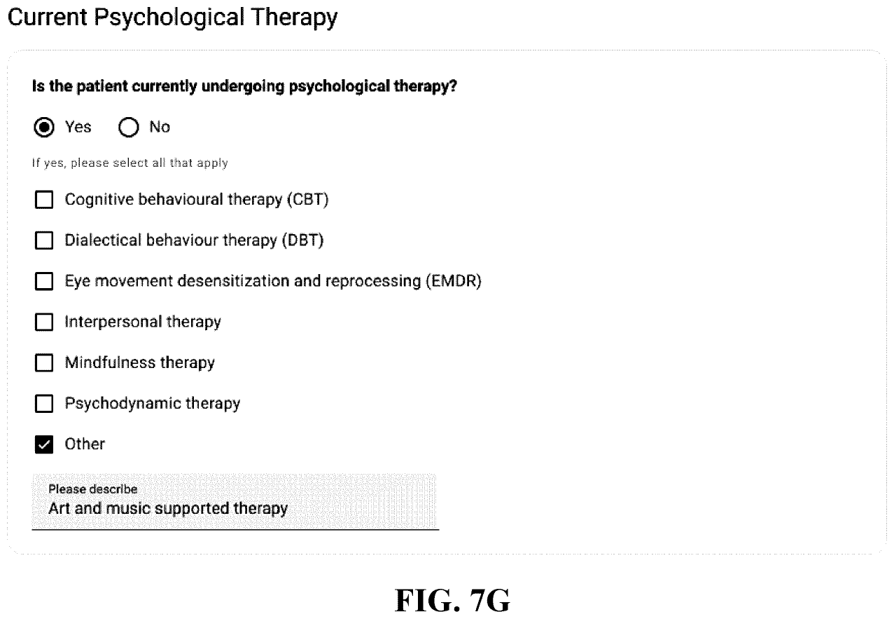

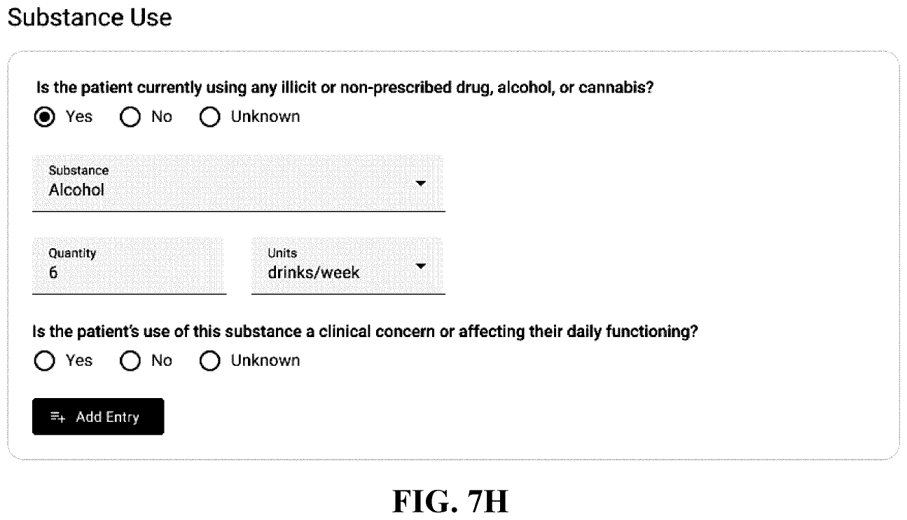

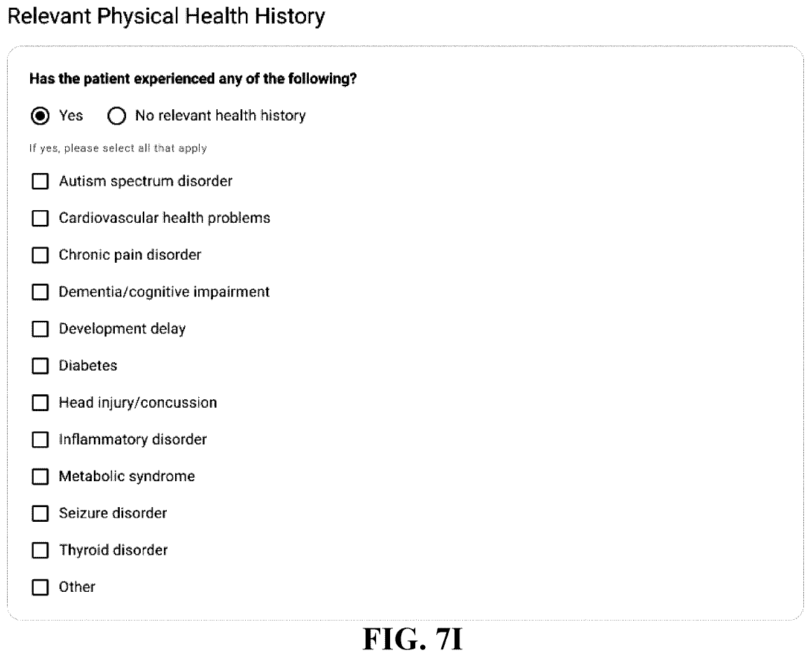

FIGS. 7A-7I depicts portions of a user interface providing a guided screening process to collect patient profile data in accordance with embodiments of the present disclosure. For example, this user interface may be utilized in whole or in part by an MA, a RAPIDS interviewer 118, a physician 114 or even a patient 116, depending on the use case. In particular, as shown in FIG. 7A, the user interface includes a sidebar with user selectable buttons corresponding to different portions of the user interface which can be navigated to by selecting the corresponding button. In the case of FIG. 7A, the button corresponding to the “Patient Information” portion of the user interface has been selected, and therefore the focus of the user interface has been configured to display the “Patient Information” portion of the user interface, which includes user interface elements that enable the user to enter some general information about the patient including date of birth, sex at birth, height, weight, and indicate whether the patient has ever had suicidal ideation or attempted suicide.

FIG. 7B depicts a “Psychiatric Diagnosis” portion of the user interface, which includes user interface elements that enable the user to enter details of a psychiatric diagnosis for the patient.

FIG. 7C depicts a “Psychiatric Scales” portion of the user interface, which includes user interface elements that enable the user to enter details of the results of psychiatric scales for the patient, such as PHQ-9.

FIG. 7D depicts a “Current Prescribed Psychopharmacological Treatments” portion of the user interface, which includes user interface elements that enable the user to enter details of any psychopharmacological treatments currently prescribed for the patient.

FIG. 7E depicts a “Past Prescribed Psychopharmacological Treatments” portion of the user interface, which includes user interface elements that enable the user to enter details of any psychopharmacological treatments previously prescribed for the patient and any reason(s) for discontinuation.

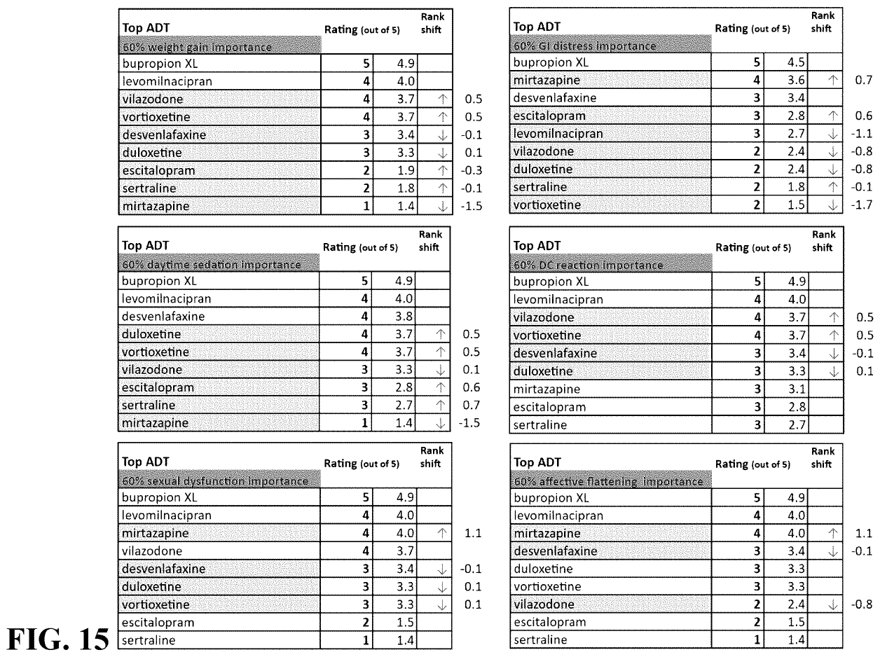

FIG. 7F depicts a “Patient Concerns” portion of the user interface, which includes user interface elements that enable the user to indicate whether there are any patient-identified complaints or clinical concerns for the patient, and, if so, what they are by selecting from a list of potential concerns. It is noted here that any concerns flagged in this portion of the user interface and/or any reasons for discontinuation of previously prescribed treatments indicated in the “Past Prescribed Psychopharmacological Treatments” may be considered by the rules logic when it comes to identifying the preferred psychopharmacological treatment options for the patient. For example, a patient concern such as weight gain or sexual dysfunction may cause a corresponding tolerability factor to be more heavily weighted in the calculation of tolerability ratings, as discussed in further detail below with reference to FIG. 15.

FIG. 7G depicts a “Current Psychological Therapy” portion of the user interface, which includes user interface elements that enable the user to indicate whether the patient is currently undergoing psychological therapy, and, if so, identify all that apply.

FIG. 7H depicts a “Substance Abuse” portion of the user interface, which includes user interface elements that enable the user to indicate whether the patient is currently using any illicit or non-prescribed drug, alcohol, or cannabis, and, if so, identify all that apply and in what quantity.

FIG. 7I depicts a “Relevant Physical Health History” portion of the user interface, which includes user interface elements that enable the user to indicate whether the patient has experienced any health history that may be relevant to diagnosis or treatment, and, if so, identify all that apply.

In some embodiments, the personalized diagnostic and treatment guidance provided by the RAPIDS service may be implemented using a rules engine, such as DROOLS, to encode a series of rules that walk the clinical user systematically through an optimized path in the application based on the supplied medical information about the patient.

In general, validation for a mental health diagnosis is arrived at by evaluating the score from the relevant standardized assessment scales (e.g., the results of the patients completing the PHQ9 for depression and GAD7 for anxiety as initial input), and based on the score results of the previously entered scales, the rules engine may trigger the request for additional standardized assessment scale(s). For example, if the PHQ9 confirms depression, then the system rules may trigger the request for completion of an MDQ assessment scale to assess for bipolar. In some cases, no standardized assessment exists for the condition. In those cases, the interviewer may use the DSM-5 criteria for the condition to complete the assessment. For example, there is no standardized assessment for determining the type of bipolar so the interviewer may use the DSM-5 criteria for bipolar to confirm and determine which type of bipolar the patient has.

In general, the recommended treatment plan is arrived at by assessing whether the patient should be on medication based on their current diagnosis and severity. If so, the pharmacological treatment rules simplify the selection of an appropriate medication as discussed earlier.

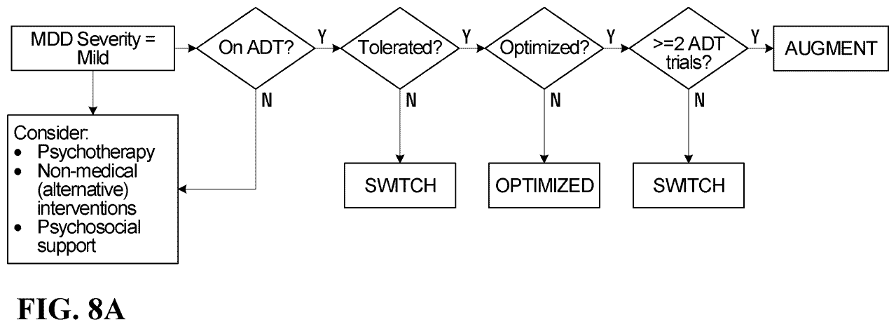

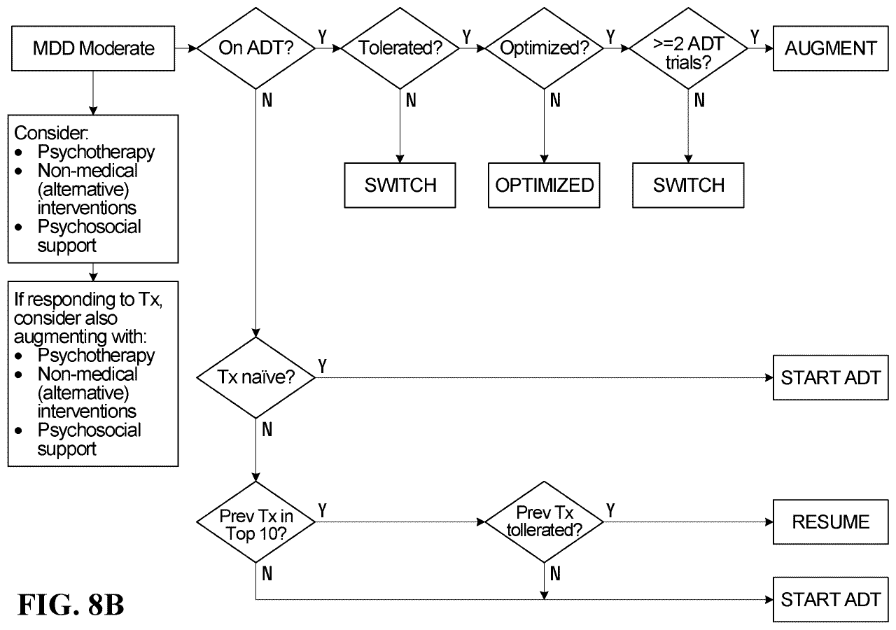

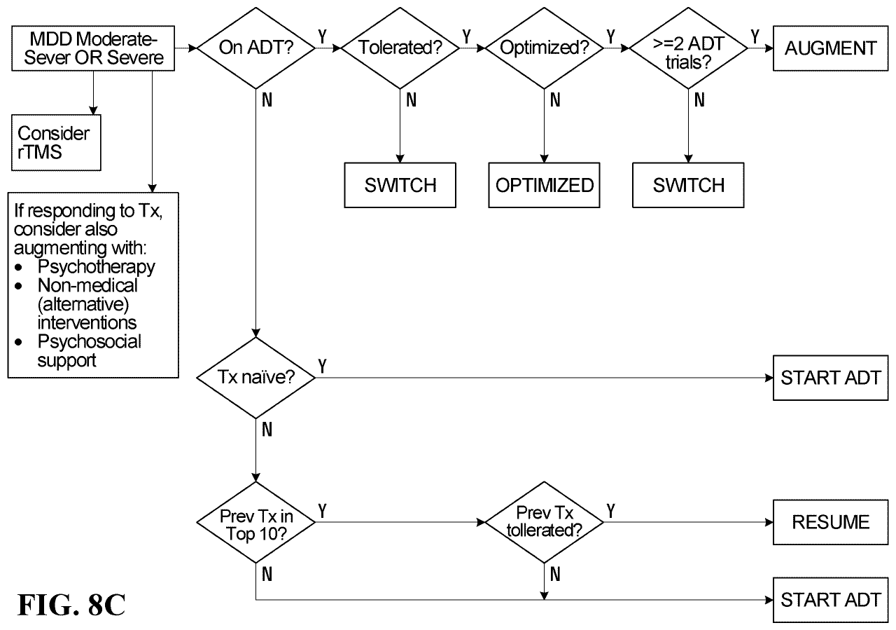

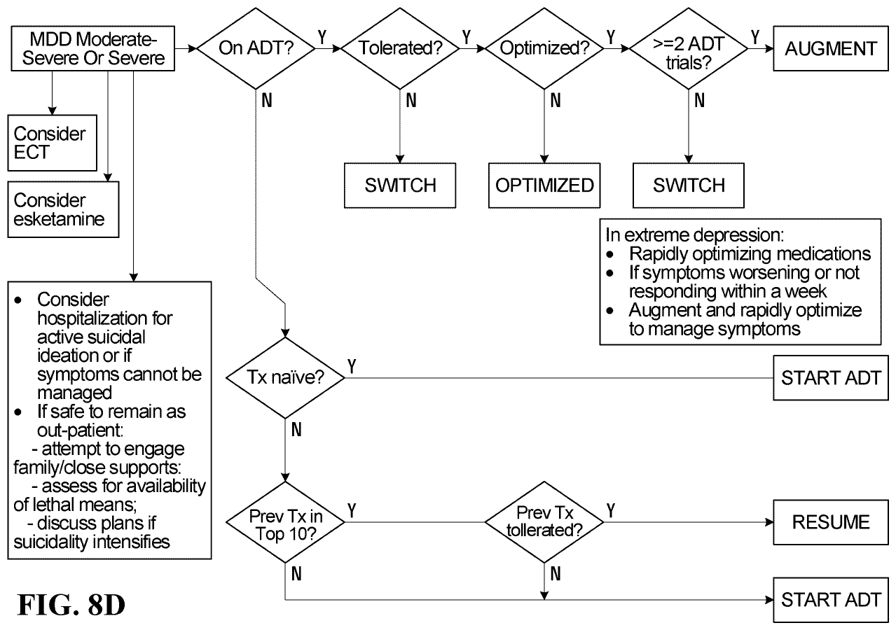

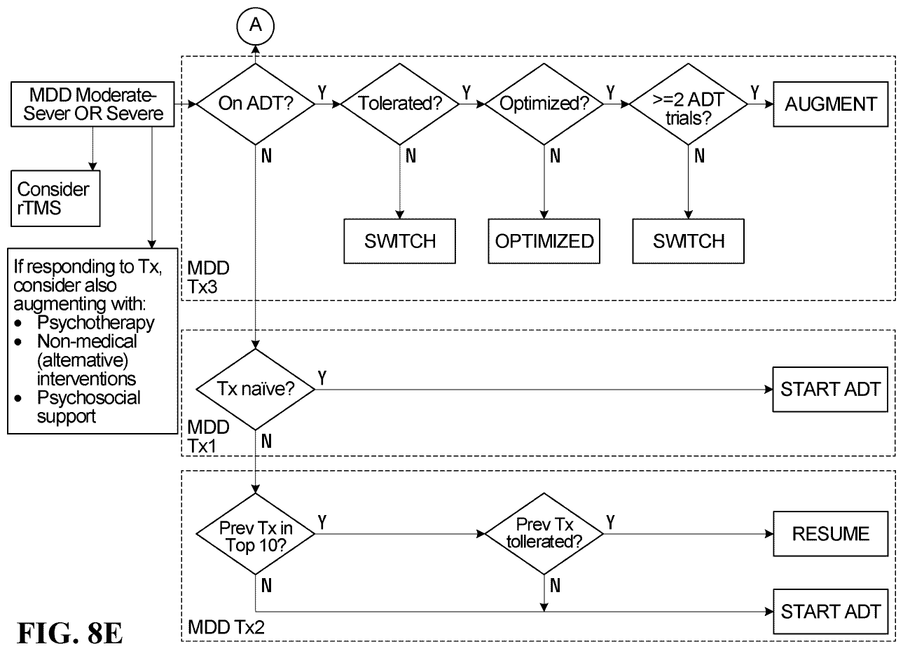

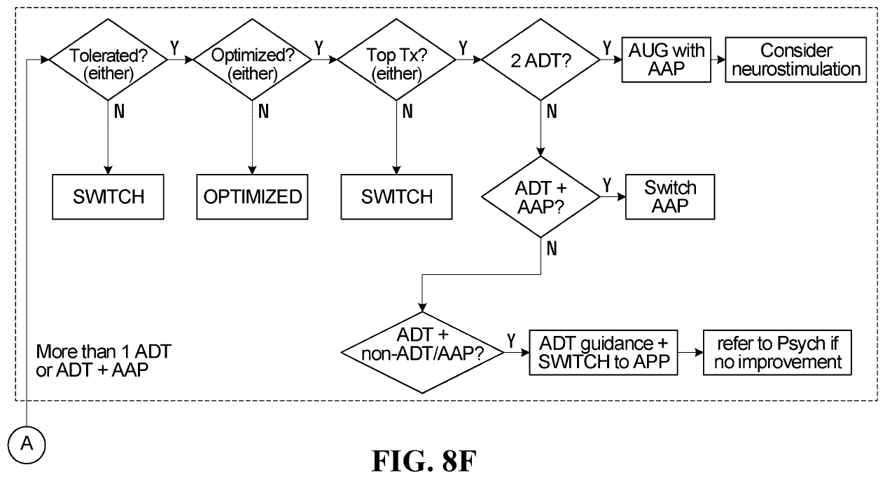

FIGS. 8A-8E depict examples of rule logic to arrive at treatment guidance for diagnoses involving MDD in accordance with embodiments of the present disclosure. In particular, FIG. 8A depicts a non-limiting example of rule logic for treating MDD with a mild severity, FIG. 8B depicts a non-limiting example of rule logic for treating MDD with a moderate severity, FIG. 8C depicts a non-limiting example of rule logic for treating MDD that is moderate-severe or severe, and FIG. 8D depicts a non-limiting example of rule logic for treating extremely severe MDD. FIG. 8E is similar to FIG. 8C in that it also depicts a non-limiting example of rule logic for treating MDD that is moderate-severe or severe. However, FIG. 8E differs from FIG. 8C in that the rule logic depicted in FIG. 8E additionally includes rule logic for the prescription of more than one antidepressant (ADT) at a time or the prescription of an ADT and an atypical antipsychotic (AAP).

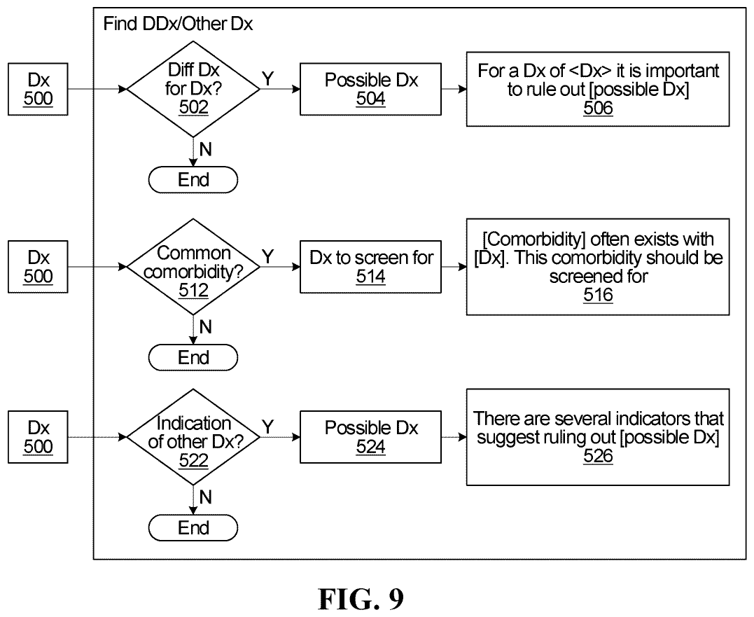

FIG. 9 depicts an example of rule logic to find differential diagnoses, other diagnoses and comorbidities for a given diagnosis in accordance with an embodiment of the present disclosure. In this example, an assessment is made at 502 as to whether a given diagnosis 500 has at least one differential diagnosis that should be ruled out. If so, each differential diagnosis is identified as a possible diagnosis as indicated at 504 and a user may be provided with guidance indicating that it is important to rule out each differential diagnosis as indicated at 506. For example, the given diagnosis 500 could be major depressive disorder, for which bipolar disorder is a differential diagnosis. In this scenario, a user may be provided with guidance indicating that for a diagnosis of major depressive disorder it is important to rule out bipolar disorder. In some cases, in addition to providing the foregoing guidance, this scenario may trigger a request for completion of an MDQ assessment scale to assess for bipolar disorder.

As shown in FIG. 9, an assessment is made at 512 as to whether the given diagnosis 500 has at least one common comorbidity that should be screened for. If so, each common comorbidity is identified as a comorbidity to screen for at 514 and a user may be provided with guidance indicating that, for each common comorbidity, the comorbidity often exists with the given diagnosis 500 and, therefore, should be screened for, as indicated at 516. For example, the given diagnosis 500 could be generalized anxiety disorder, for which major depressive disorder is a common comorbidity. In this scenario, a user may be provided with guidance indicating that major depressive disorder often exists with generalized anxiety disorder and should be screened for. In some cases, in addition to providing the foregoing guidance, this scenario may trigger a request for completion of a PHQ-9 assessment scale to assess for major depressive disorder.

As shown in FIG. 9, an assessment may also be made at 522 as to whether there are any indicators of other diagnoses in addition to diagnosis 500. For example, this could involve assessing the patient profile data to look for indicators in the patient profile data that are suggestive of a diagnosis that has not already been considered (e.g. not a differential diagnosis or common comorbidity). This may include looking for indicators such as a family history of disorders, age of onset, specific symptoms or groups of symptoms, etc. Each other diagnosis for which one or more indicators is present may be identified as a possible diagnosis to screen for, as indicated at 524, and a user may be provided with guidance indicating that, for each other diagnosis, there are indicators that suggest ruling out the other diagnosis, as indicated at 526.

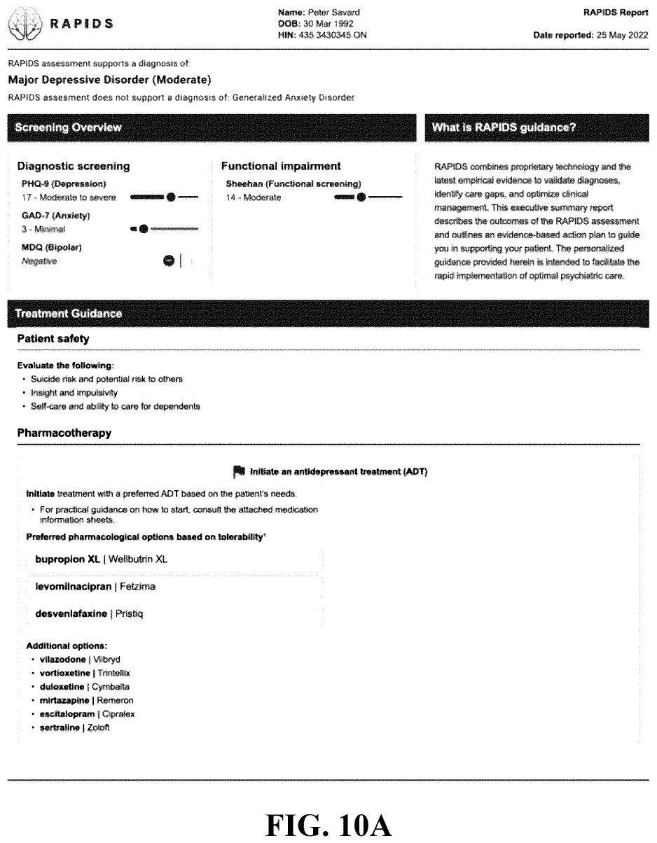

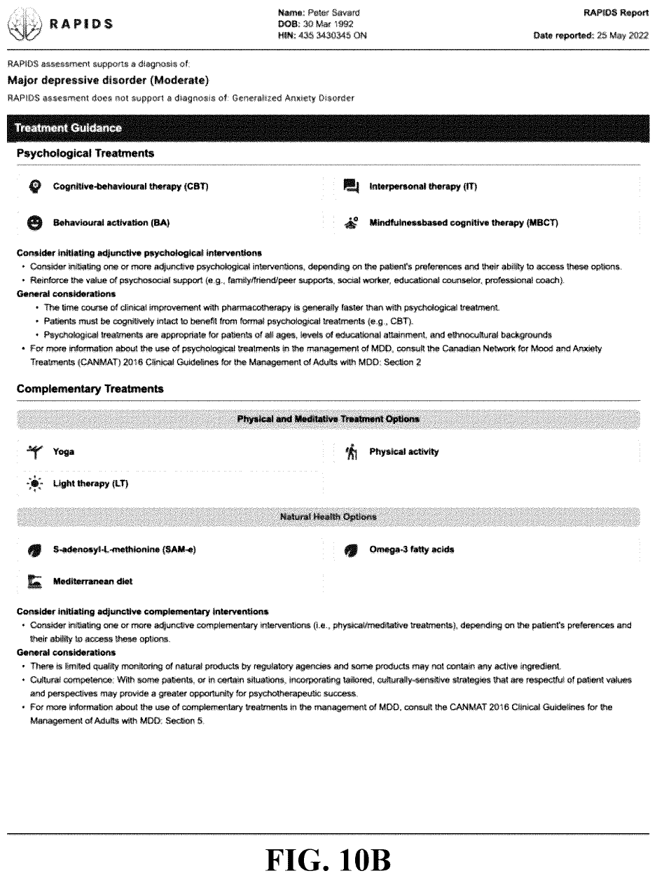

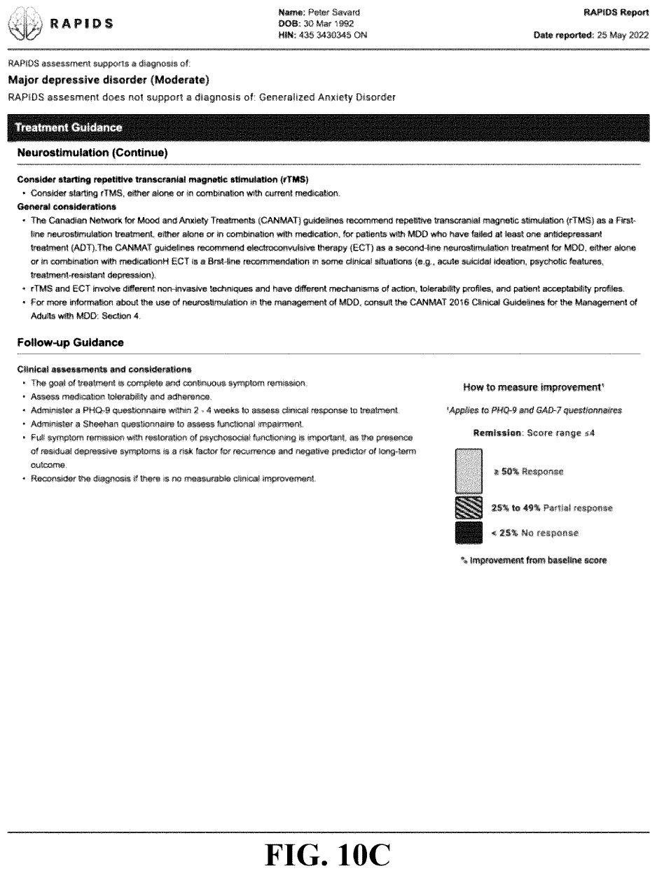

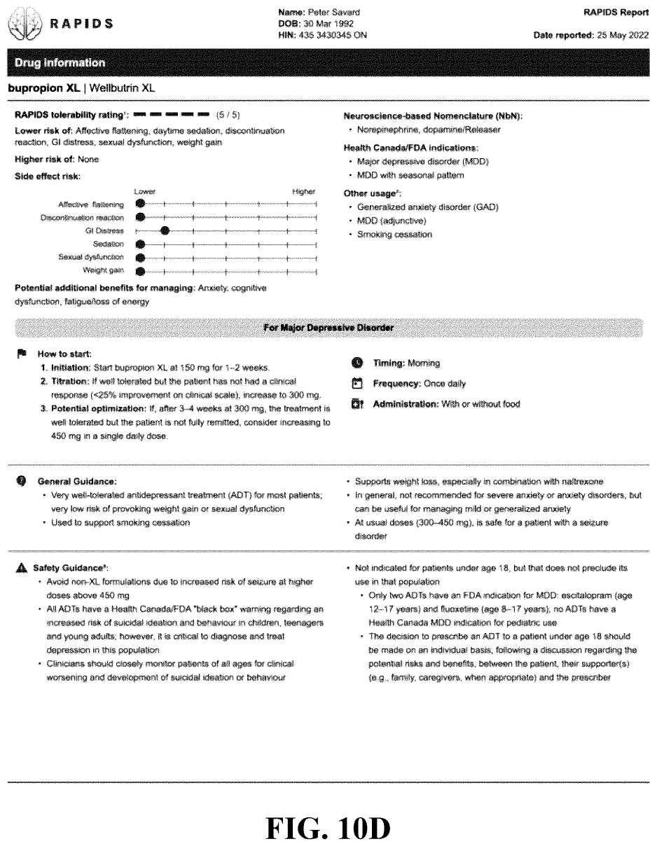

FIGS. 10A-10D depict example pages of a report that includes personalized diagnostic and treatment guidance for a particular patient in accordance with an embodiment of the present disclosure. The example report depicted in FIGS. 10A-10D includes three main sections, namely a screening overview section, a treatment guidance section and a drug information section. In this case, the heading of the report indicates that the RAPIDS assessment supports a diagnosis of MDD. The screening overview section provides a summary of the screening results, which support the indicated diagnosis, and the treatment guidance section sets out the recommended treatment plan for that diagnosis for this particular patient. The treatment guidance section of the report includes six main components, namely a patient safety component, a pharmacotherapy component, a psychological treatments component, a complementary treatments component, a neurostimulation component and a follow-up guidance component.

The patient safety component provides guidance regarding potential issues that should be evaluated before and during any implementation of the treatment guidance.

The pharmacotherapy component includes an ordered subset of preferred pharmacological treatment options based on tolerability. In this case, the diagnosis does not include any comorbid disorders, such as insomnia. However, it is noted that in a case where the patient is diagnosed with a comorbid disorder, the pharmacotherapy component of the report may also include a second ordered subset of preferred pharmacological treatment options for treatment of the second comorbid health disorder, e.g., an ordered subset of preferred pharmacological treatment options for treatment of comorbid insomnia.

The psychological treatments component of the treatment guidance provides guidance on psychological treatments for MDD, including CBT, IT, BA and MBCT.

The complementary treatments component of the treatment guidance provides complementary and alternative (non-medical) treatments for MDD.

In this example, the neurostimulation component of the report indicates that starting neurostimulation should be considered based on the patient's diagnosis and the severity. For example, neurostimulation is an option for moderate to severe depression, and not for anxiety or moderate depression.

Finally, the follow-up guidance component of the report provides guidance for follow-up assessments of the patient's response to treatment of the diagnosed disorders.

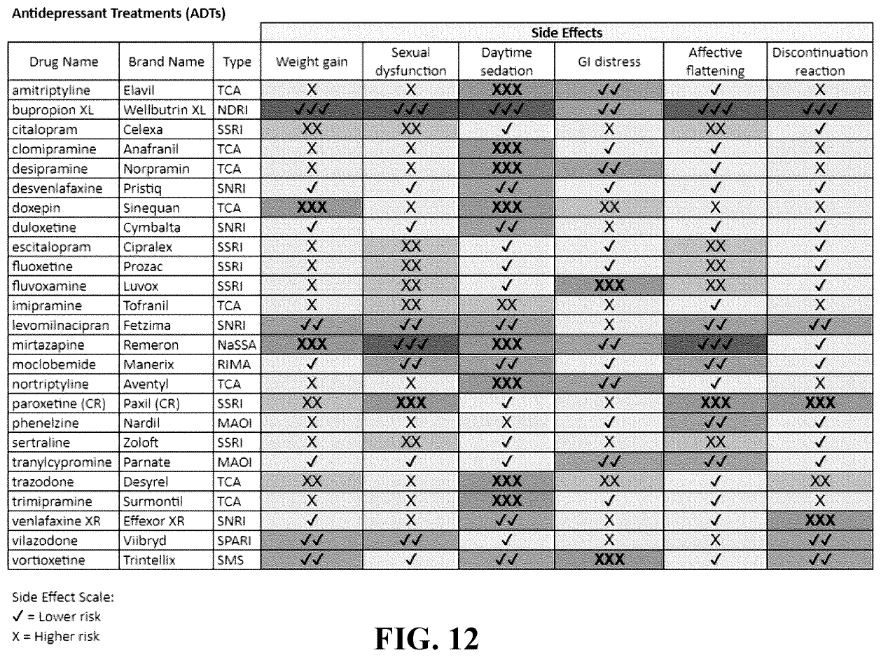

In this example, the drug information component of the report includes drug information for each pharmacological treatment option for the treatment of MDD, including an indication of a single tolerability rating out of 5 for the preferred pharmacological treatment options. For example, FIG. 10D includes a drug information summary for bupropion XL/Wellbutrin XL, which is indicated to be the most preferred pharmacological option based on tolerability in the pharmacotherapy component of the report shown in FIG. 10A. In this case, the drug information for bupropion XL/Wellbutrin XL indicates that it has a tolerability rating of 5 out of 5. Further details related to such tolerability ratings are discussed in further detail later on with reference to FIGS. 12-16

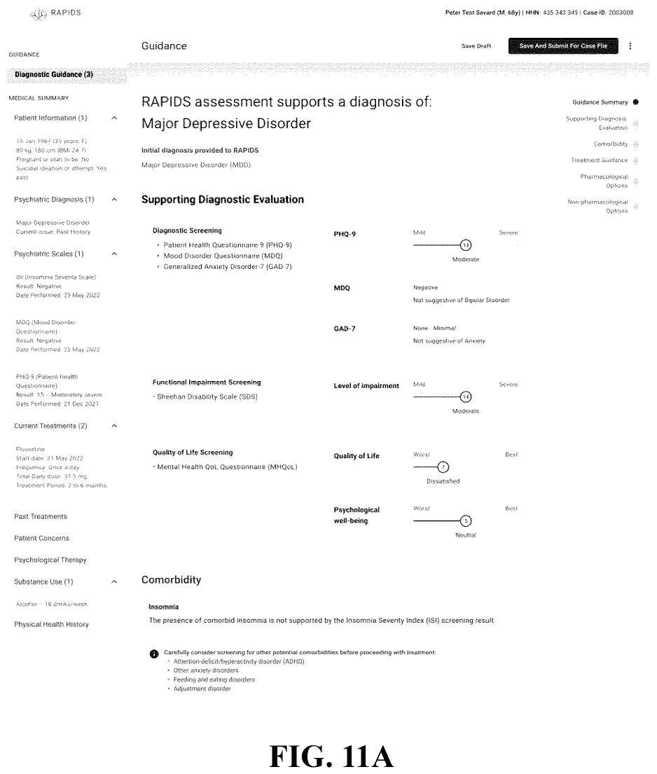

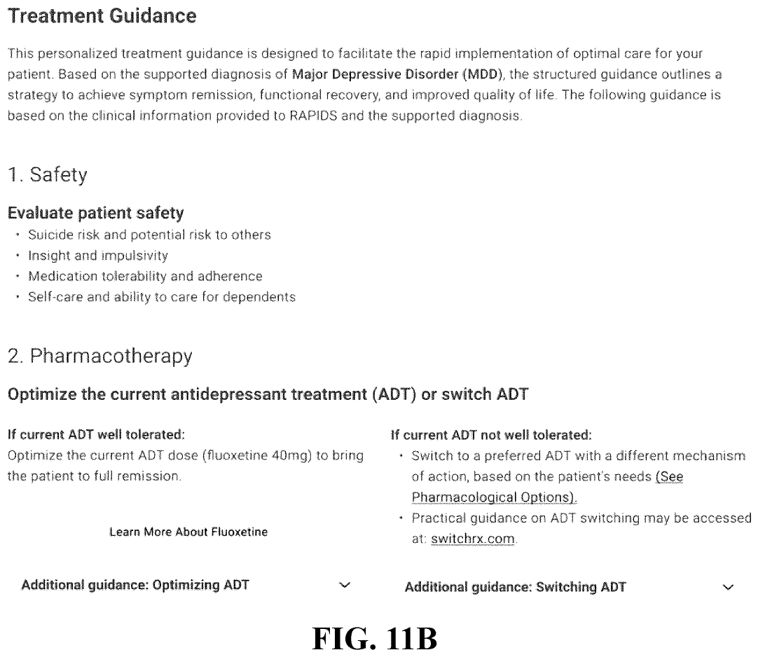

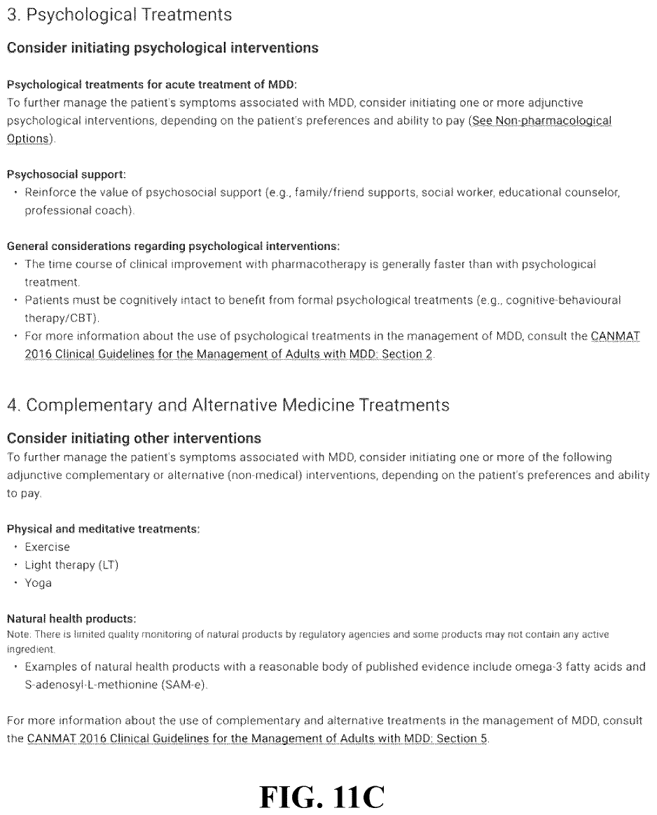

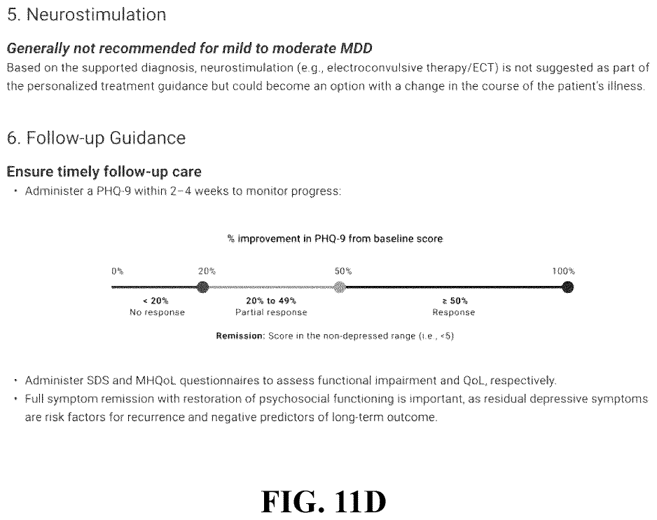

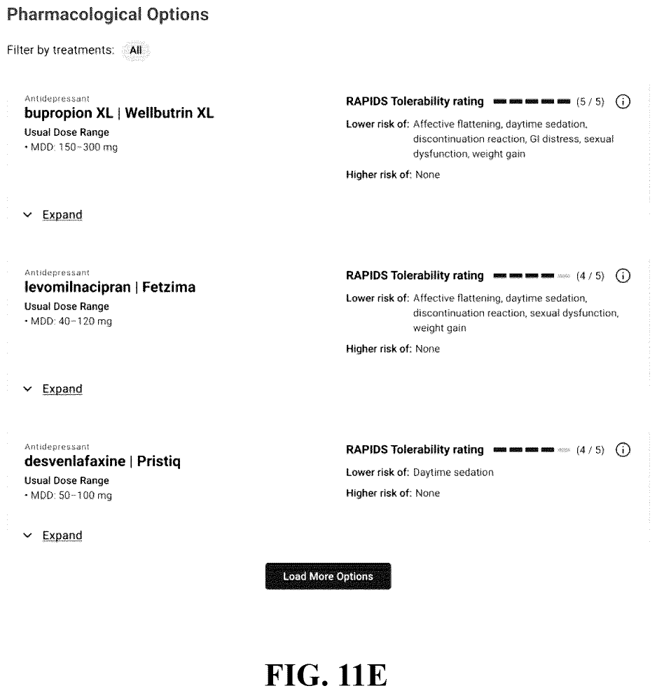

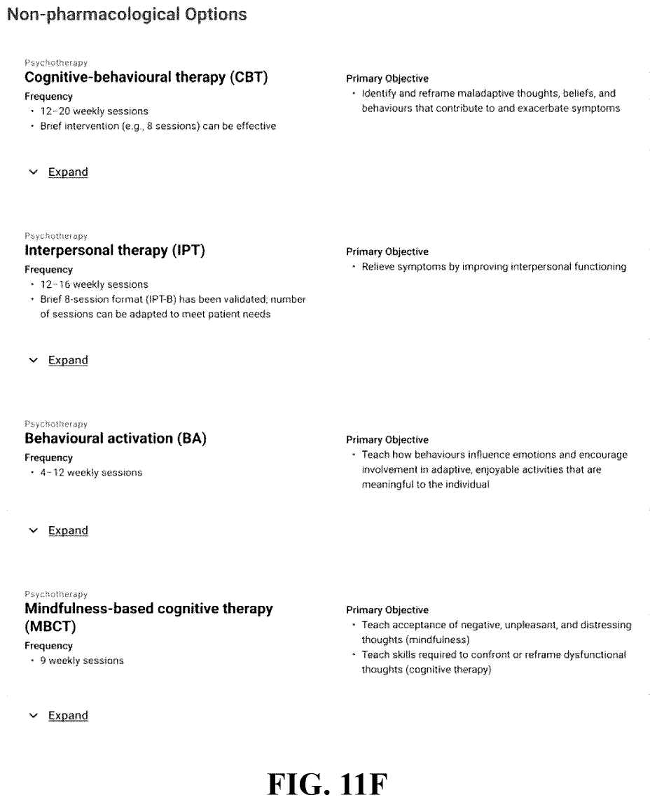

FIGS. 11A-11F depict portions of a user interface providing another example of an electronic report that includes personalized diagnostic and treatment guidance in accordance with an embodiment of the present disclosure. For example, this user interface may be utilized in whole or in part by an insurance claims manager, a physician 114 or even a patient 116, depending on the use case. As shown in FIG. 11A, the user interface includes a sidebar with user selectable buttons corresponding to different portions of the user interface which can be navigated to by selecting the corresponding button. In the case of FIG. 11A, the button corresponding to the “Diagnostic Guidance” portion of the user interface has been selected, and therefore the focus of the user interface has been configured to display the “Guidance” portion of the user interface, which includes user interface elements that enable the user to review the supporting diagnostic evaluation that, in this case, supports a diagnosis of MDD and is consistent with the initial diagnosis that was provided to the RAPIDS service.

FIGS. 11B, 11C and 11D depict the “Treatment Guidance” portion of the user interface, which, similar to the preceding example includes five main components, namely a patient safety component, a pharmacotherapy component, a psychological treatments component, a neurostimulation component and a follow-up guidance component. In this case, the additional guidance regarding optimizing the currently prescribed ADT and the additional guidance regarding switching ADTs are accessible through selection of user-selectable user interface elements, as shown in FIG. 11B.

FIG. 11E depicts a “Pharmacological Options” portion of the user interface, which provides information about each of the three preferred pharmacological treatment options, including a single tolerability rating out of five for each preferred option.

FIG. 11F depicts a “Non-pharmacological Options” portion of the user interface, which in this case provides information about non-pharmacological treatment options for MDD, including CBT, IPT, BA and MBCT.

FIG. 12 depicts an example of a table of medication side effect gradings that may be used to determine tolerability ratings for the listed medications in accordance with an embodiment of the present disclosure. In this non-limiting example, each medication is assigned a grade or score for each of a plurality of side effects. In this specific example, there are seven possible grades for each side effect. The seven possible grades are defined as follows from high to low (or good to bad): three checks, two checks, one check, no checks, one X, two Xs, three Xs.

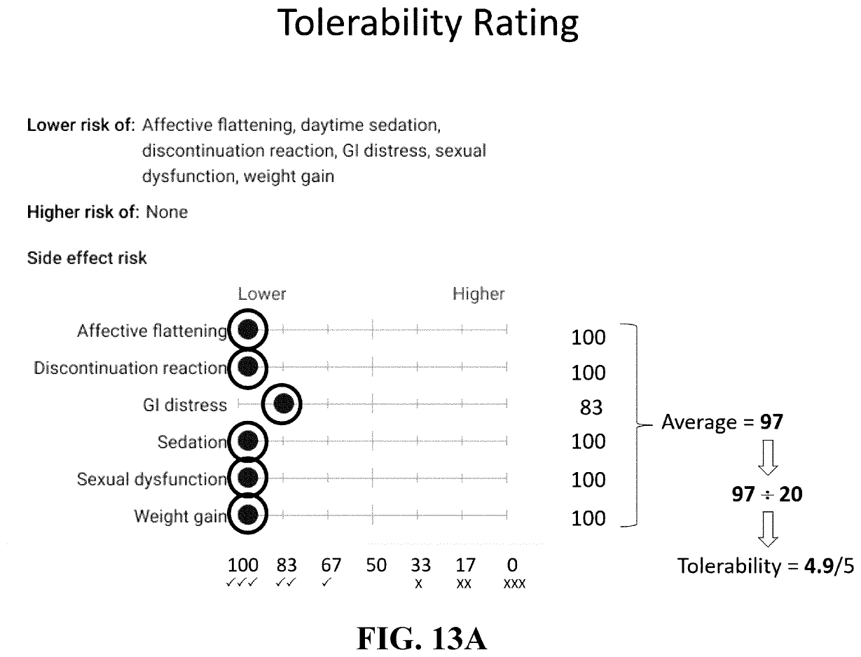

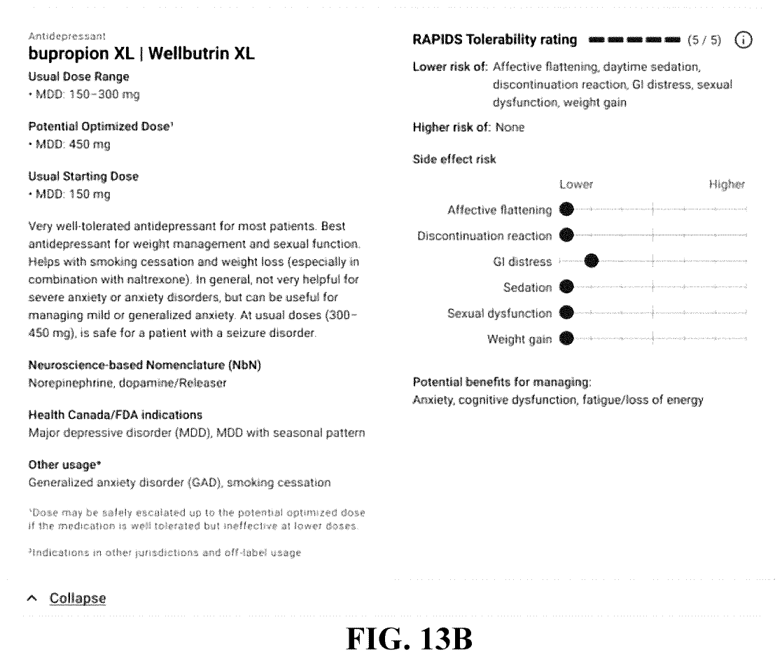

FIGS. 13A and 13B depict an example of calculating a single tolerability rating for a medication as a function of multiple tolerability factors based on the side effect gradings of FIG. 12. In particular, as shown in FIG. 13A, one non-limiting example of calculating a single tolerability rating would be to assign the grades equally incremented numerical values from 0 to 100, add up the total of the numerical values for all of the graded side effects considered in the tolerability rating and divide the total by the number of side effects considered in the tolerability rating in order to determine the average numerical rating out of 100. In the example shown in FIG. 13A, which is for the ADT bupropion XL/Wellbutrin XL, the rating out of 100 by this method is 97, which when divided by 20 can be converted to a rating of 4.9 out of 5 (rounded up to one decimal place) or 5 out of 5 (rounded up to the nearest 0.5). For example, FIG. 13B depicts the additional information about bupropion XL/Wellbutrin XL, including a 5/5 tolerability rating, that may be displayed when the “Expand” icon is selected for the bupropion XL/Wellbutrin XL pharmacological option in the “Pharmacological Options” portion of the user interface shown in FIG. 11E. It should be noted that this specific method of calculating a tolerability rating is merely one example of how a single tolerability rating may be calculated as a function of multiple tolerability factor ratings.

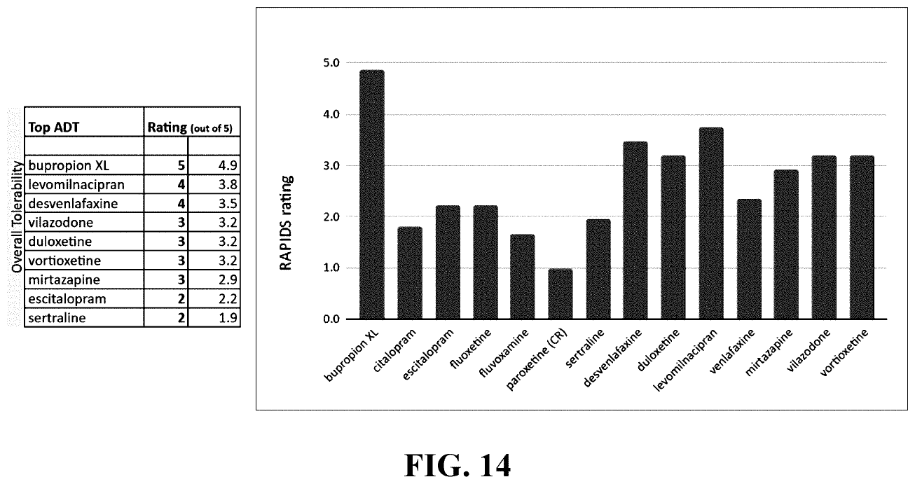

FIG. 14 depicts a table and a corresponding chart that show the respective single tolerability rating expressed as a rating out of five for nine of the medications listed in the table of FIG. 12 having the highest single tolerability ratings in accordance with an embodiment of the present disclosure.

In some embodiments, the tolerability rating may be personalized for a particular patient by unequally weighting the tolerability factors, e.g., by more heavily weighting one or more of the tolerability factors that are identified as being of particular importance to the patient.

FIG. 15 depicts six tables showing changes in the tolerability ratings of the medications listed in the table of FIG. 14 for six different scenarios in which the tolerability factors related to weight gain, GI distress, sexual dysfunction, daytime sedation, discontinuation reaction, and affective flattening, respectively, are more heavily weighted in the calculation of the tolerability rating in accordance with an embodiment of the present disclosure. In particular, in this example, rather than having each of the tolerability factors or side effects equally weighted in the calculation, one of the tolerability factors is weighted at 60% and the remaining weighting of 40% is equally divided among the remaining tolerability factors. For example, in the top left table of FIG. 15, the tolerability factor corresponding to the weight gain side effect is given a 60% weighting and each of the other five tolerability factors (sexual dysfunction, daytime sedation, GI distress, affective flattening and discontinuation reaction) is give an 8% weighting. In this scenario, a medication with a high risk of weight gain, such as mirtazapine/Remeron, is reduced from a rating of 3 to a rating of 1. However, even more significantly, a medication with a low risk of weight gain, such as desvenlafaxine/Pristiq, can be bumped out of the top three by a medication with an even lower risk of weight gain, such as vilazodone/Viibryd.

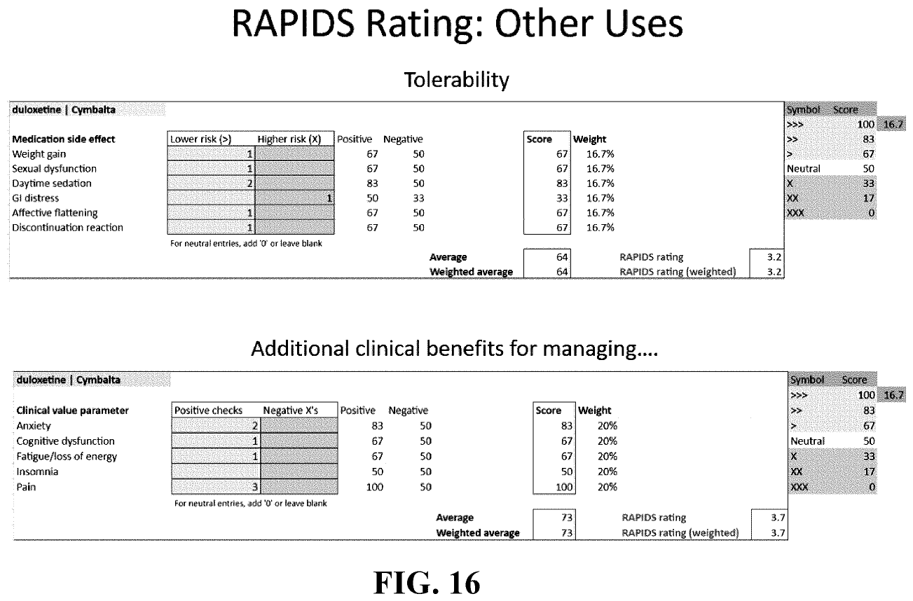

FIG. 16 illustrates an example of how an additional clinical benefits rating may be calculated for a medication in a manner similar to that used to calculate a tolerability rating for the medication in accordance with an embodiment of the present disclosure. For example, as shown in FIG. 16, a medication that is primarily for the treatment of one disorder may be assigned an additional benefit rating as an indication of its ability to provide additional clinical benefits for managing one or more additional conditions/disorders. In particular, in this example, the medication duloxetine/Cymbalta may be used to treat MDD, but also has additional clinical benefits in managing other conditions/disorders, such as generalized anxiety disorder, fibromyalgia, neuropathic pain and central sensitization. As such, in some embodiments, rather than ranking pharmacological treatment options based on a tolerability rating, pharmacological treatment options may also or instead be ranked based on an additional clinical benefits rating. For example, the two tables in FIG. 16 show non-limiting examples of how a tolerability rating and an additional clinical benefits rating may be calculated for duloxetine/Cymbalta. In some embodiments, the additional clinical benefit factors (e.g., anxiety, cognitive dysfunction, fatigue/loss of energy, insomnia and pain in this particular non-limiting example) that the overall additional clinical benefits rating is based upon may be unequally weighted. For example, for a patient with both MDD and neuropathic pain, the rating for the additional clinical benefit factor corresponding to pain management may be more heavily weighted. For example, as shown in the lower table in FIG. 16, with an equal weighting of each of the additional clinical benefit factors, the overall additional clinical benefit factor rating for duloxetine/Cymbalta is 3.7/5. However, if the pain management factor is given a 60% weighting and the remaining 40% weighting is equally divided among the remaining four factors, the overall additional clinical benefit factor rating for duloxetine/Cymbalta rises to 4.3/5. In addition or instead, in some embodiments the tolerability rating and the additional clinical benefits rating may be combined into a composite rating. For example, the overall additional clinical benefit factor rating of 3.7/5 and the overall tolerability rating of 3.2/5 may be combined and averaged to a composite score of 3.5/5 (rounded up to 1 decimal place). In still further embodiments, the two ratings may be unequally weighted with more weight being given to one rating or the other depending on patient concerns or comorbidities, for example.

Many of the foregoing examples have been described in the context of utilizing a rules-based engine to implement rules logic for providing personalized diagnostic and treatment guidance. The algorithms underlying such rule and knowledge based systems are deterministic in nature. And yet, there is inherent uncertainty in different aspects of the medical domain, (e.g., the manifestation of symptoms, the interpretation of a psychometric question by a patient, the response variability to a specific treatment). In some embodiments, such knowledge-based systems/platform may be augmented with a data-driven probabilistic approach.

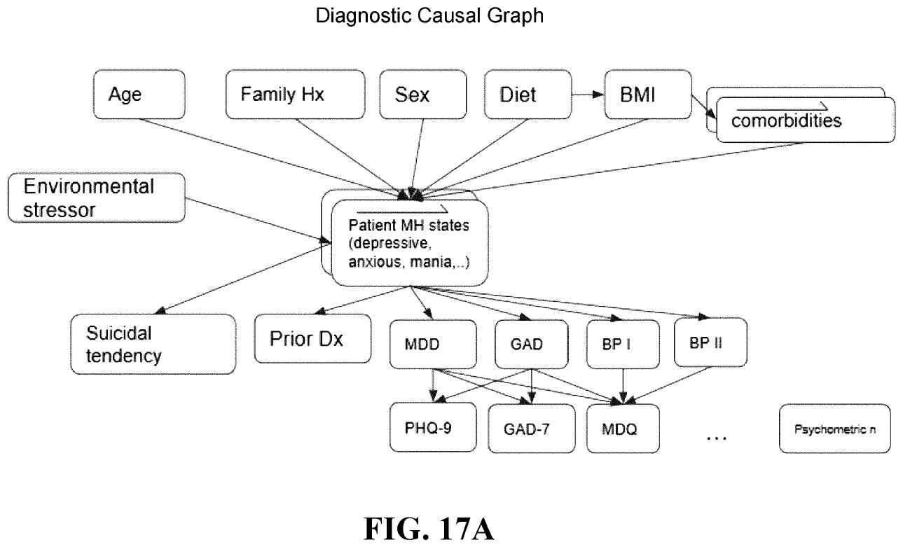

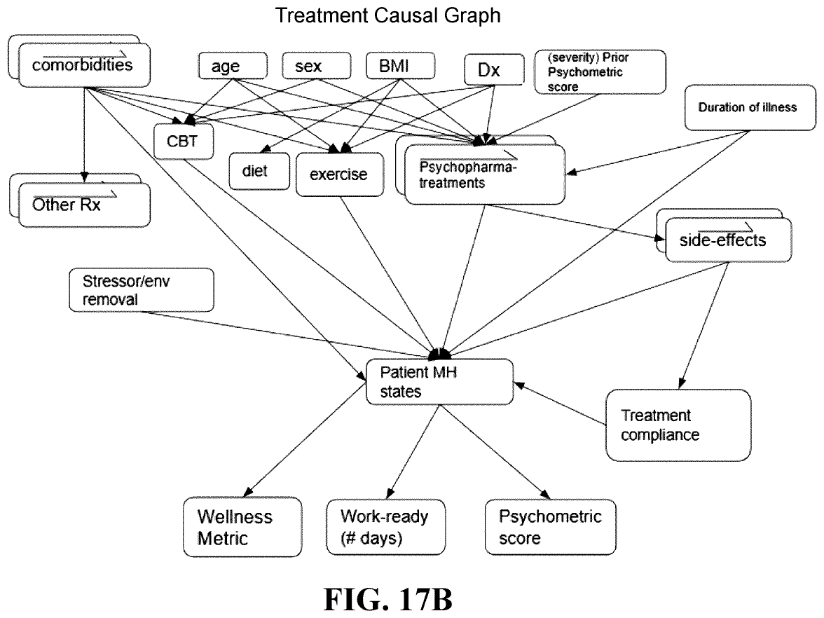

For example, in some embodiments a Bayesian Network may be defined that captures the influence or causal relationships amongst the conceptual entities (signs, symptoms, risk factors, manifestations, conditions, medical tests, treatments, comorbidities, etc.) that surface in a Mental Health diagnostic and treatment clinical context. A Bayesian Network is a probabilistic graphical model that represents a set of variables and their conditional dependencies via a directed acyclic graph (DAG). Each node in the graph has a Conditional Probability Table (CPT). As evidence comes in (e.g. a medical test, or a new symptom) the BN is conditioned on the relevant nodes and the belief updates are propagated through the network yielding revised probabilities. For example, FIGS. 17A and 17B depict examples of causal graphs for diagnosis and treatment respectively, in accordance with embodiments of the present disclosure. In particular, FIG. 17A depicts an example of a diagnostic causal graph in accordance with an embodiment of the present disclosure, and FIG. 17B depicts an example of a treatment causal graph in accordance with an embodiment of the present disclosure.

The networks depicted in FIGS. 17A and 17B could potentially be combined but have been kept separate in these illustrated examples in order to denote the 2-step process of diagnosis and treatment.

The inventors of the present disclosure have recognized that it is important for a clinician to think mechanistically when considering optimal treatment options for a patient. With that in mind, the expansion of a Bayesian Network to model the brain functions and chemistry implicated in mood disorders and their treatment is contemplated. In doing so, “what-if analyses” can be supported in clinical reasoning scenarios, which enables progress towards a mental health “digital twin”.

Although the present disclosure describes methods and processes with steps in a certain order, one or more steps of the methods and processes may be omitted or altered as appropriate. One or more steps may take place in an order other than that in which they are described, or even all simultaneously, as appropriate. For example, referring again to the flowchart depicted in FIG. 4, in some implementations a rules-based engine may be used to implement rules logic for providing the personalized diagnostic and treatment guidance through a non-linear process based on any combination of a set of factors, whereby a unique set of factors, such as assessment scales provided, health disorder, treatments, comorbidities, etc., returns a unique version of a report.

Although the present disclosure is described, at least in part, in terms of methods, a person of ordinary skill in the art will understand that the present disclosure is also directed to the various components for performing at least some of the aspects and features of the described methods, be it by way of hardware components, software or any combination of the two. Accordingly, certain technical solutions of the present disclosure may be embodied in the form of a software product. A suitable software product may be stored in a pre-recorded storage device or other similar non-volatile or non-transitory computer readable medium, for example. The software product includes instructions tangibly stored thereon that enable a processing device (e.g., a microprocessor) to execute examples of the methods disclosed herein.

The present disclosure may be embodied in other specific forms without departing from the subject matter of the claims. The described example embodiments are to be considered in all respects as being only illustrative and not restrictive. Selected features from one or more of the above-described embodiments may be combined to create alternative embodiments not explicitly described, features suitable for such combinations being understood within the scope of this disclosure.

Although the systems, devices and processes disclosed and shown herein may comprise a specific number of elements/components, the systems, devices and assemblies could be modified to include additional or fewer of such elements/components. For example, although any of the elements/components disclosed may be referenced as being singular, the embodiments disclosed herein could be modified to include a plurality of such elements/components. The subject matter described herein intends to cover and embrace all suitable changes in technology.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or more and the singular also includes the plural unless it is obvious that it is meant otherwise.

Further, use of the term “plurality” is meant to convey “more than one” unless expressly stated to the contrary.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Circuitry, as used herein, may be analog and/or digital, components, or one or more suitably programmed microprocessors and associated hardware and software, or hardwired logic. Also, “components” may perform one or more functions. The term “component,” may include hardware, such as a processor, an application specific integrated circuit (ASIC), or a field programmable gate array (FPGA), or a combination of hardware and software. Software includes one or more computer executable instructions that when executed by one or more components cause the component to perform a specified function. It should be understood that the algorithms described herein are stored on one or more non-transitory memory. Exemplary non-transitory memory includes random access memory, read only memory, flash memory or the like. Such non-transitory memory may be electrically based or optically based.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 90%, or at least 91%, or at least 92%, or at least 93%, or at least 94%, or at least 95%, or at least 96%, or at least 97%, or at least 98%, or at least 99%, of the referenced dimension or measurement.

1. A computer-implemented method for generating personalized diagnostic and treatment guidance, the method being executed by one or more processors and comprising:
obtaining patient profile data for a patient, the patient profile data comprising a result of at least one evaluation test or interview of the patient;
determining, based on the patient profile data, diagnostic guidance for the patient, the diagnostic guidance for the patient identifying a primary health disorder of the patient and a second comorbid health disorder of the patient;
determining, based on the patient profile data and the diagnostic guidance for the patient, personalized treatment guidance to address treatment of the primary health disorder of the patient and treatment of the second comorbid health disorder of the patient, the personalized treatment guidance comprising:
a pharmacotherapy component identifying an ordered listing of pharmacological treatment options, each pharmacological treatment option corresponding to a different pharmaceutical product for treatment of the primary health disorder, wherein ordering of the pharmacological treatment options in the ordered listing is based on a ranking of the pharmacological treatment options for treatment of the primary health disorder based on the patient profile data and the diagnostic guidance for the patient;
generating an electronic report, the report comprising:
an indication of the diagnostic guidance for the patient;
an indication of the personalized treatment guidance to address treatment of the primary health disorder of the patient and treatment of the second comorbid health disorder of the patient.

2. (canceled)

3. The method of claim 1, wherein the ordered listing of pharmacological treatment options includes only a partial subset of the highest ranked pharmacological treatment options corresponding to pharmaceutical products approved or commonly used off-label by medical specialists for treatment of the primary health disorder.

4. The method claim 1, wherein the pharmacotherapy component of the personalized treatment guidance for the patient includes pharmacological treatment guidance providing at least one of the following:
guidance for starting a pharmacological treatment option from among the ordered listing of pharmacological treatment options;
guidance for maintaining a current pharmacological treatment option identified in the patient profile data;
guidance for stopping a current pharmacological treatment option identified in the patient profile data;
guidance for optimizing a dosage of a current pharmacological treatment option identified in the patient profile data;
guidance for switching from a current pharmacological treatment option to a different pharmacological treatment option from among the ordered listing of pharmacological treatment options; and
guidance for augmenting a current pharmacological treatment option with another different pharmacological treatment option from among the ordered listing of pharmacological treatment options.

5. The method of claim 1, wherein determining the personalized treatment guidance based on the patient profile data and the diagnostic guidance for the patient is performed by the one or more processors utilizing a rules engine to process the patient profile data and the diagnostic guidance relative to a rule set.

6. The method of claim 5, wherein the rule set is configured to implement empirically validated treatment guidance.

7. (canceled)

8. The method of claim 1, wherein the pharmacotherapy component of the personalized treatment guidance comprises:
a first ordered listing of pharmacological treatment options for treatment of the primary health disorder, each pharmacological treatment option of the first ordered listing corresponding to a different pharmaceutical product for treatment of the primary health disorder, wherein the ordering of the pharmacological treatment options in the first ordered listing is based on a ranking of the pharmacological treatment options for treatment of the primary health disorder based on the patient profile data and the diagnostic guidance for the patient; and
a second ordered listing of pharmacological treatment options for treatment of the second comorbid health disorder, each pharmacological treatment option of the second ordered listing corresponding to a different pharmaceutical product for treatment of the second comorbid health disorder.

9. The method of claim 8, wherein the ordering of the pharmacological treatment options in the second ordered listing is based on a ranking of the pharmacological treatment options for treatment of the second comorbid health disorder based on the patient profile data and the diagnostic guidance for the patient.

10. The method of claim 8, wherein the ordering of the pharmacological treatment options for treatment of the primary health disorder in the first ordered listing considers both treatment of the primary health disorder and treatment of the second comorbid health disorder.

11. The method of claim 8, wherein the ordering of the pharmacological treatment options for treatment of the secondary comorbid health disorder in the second ordered listing considers both treatment of the secondary comorbid health disorder and treatment of the primary health disorder.

12. The method of claim 1, wherein determining the diagnostic guidance for the patient comprises:
identifying multiple health disorders of the patient based on the patient profile data;
identifying one health disorder of the multiple health disorders of the patient as the primary health disorder of the patient based on a hierarchy for treatment of health disorders;
identifying another health disorder of the multiple health disorders of the patient as the second comorbid health disorder of the patient based on the hierarchy for treatment of health disorders.

13. The method of claim 12, wherein determining the diagnostic guidance for the patient further comprises identifying each remaining health disorder of the multiple health disorders of the patient as a further comorbidity to the primary health disorder based on the hierarchy for treatment of health disorders.

14. The method of claim 13, wherein the personalized treatment guidance addresses treatment of the primary health disorder and treatment of the health disorders identified as comorbidities to the primary health disorder in accordance with a priority based on the hierarchy for treatment of health disorders.

15. The method of claim 1, wherein the ranking of the pharmacological treatment options is further based on a respective tolerability rating for each pharmacological treatment option.

16. The method of claim 15, wherein, for each pharmacological treatment option, the respective tolerability rating is a single rating that is a function of respective ratings for a plurality of tolerability factors for that pharmacological treatment option.

17. The method of claim 16, wherein the plurality of tolerability factors for a given pharmacological treatment option correspond to potential side effects from an associated pharmacological treatment class, and wherein the respective rating for a given tolerability factor indicates a likelihood of experiencing the side effect corresponding to that tolerability factor.

18. (canceled)

19. (canceled)

20. The method of claim 15, wherein, for each pharmacological treatment option, the respective tolerability rating is a weighted function of the respective ratings for the plurality of tolerability factors for that pharmacological treatment option, wherein respective weightings of the respective ratings for the plurality of tolerability factors are based on at least one of:
patient-identified complaints or clinical concerns identified in the patient profile data;
patient health history identified in the patient profile data; or
one or more comorbidities identified in the diagnostic guidance.

21. The method of claim 15, wherein the indication of the personalized treatment guidance in the electronic report comprises, for each pharmacological treatment option in the ordered listing of pharmacological treatment options, an indication of the respective tolerability rating for that pharmacological treatment option.

22. (canceled)

23. (canceled)

24. The method of claim 1, wherein determining the diagnostic guidance for the patient based on the patient profile data comprises:
for a given diagnosis, determining one or more differential diagnoses to rule out;
assessing the given diagnosis and the one or more differential diagnoses based on the patient profile data to identify a supported diagnosis;
identifying the supported diagnosis as the diagnostic guidance for the patient;
for the supported diagnosis, assessing one or more common comorbidities of the supported diagnosis based on the patient profile data to determine whether a given comorbidity is supported by the patient profile data; and
for each comorbidity that is determined to be supported by the patient profile data, identifying the comorbidity in the diagnostic guidance for the patient.

25.-37. (canceled)

38. A device, comprising:
one or more processors;
memory; and
one or more programs, wherein the one or more programs are stored in the memory and configured to be executed by the one or more processors, the programs including:
instructions for obtaining patient profile data for a patient, the patient profile data comprising a result of at least one evaluation test or interview of the patient;
instructions for determining, based on the patient profile data, diagnostic guidance for the patient, the diagnostic guidance identifying a primary health disorder of the patient and a second comorbid health disorder of the patient;
instructions for determining, based on the patient profile data and the diagnostic guidance for the patient, personalized treatment guidance to address treatment of the primary health disorder of the patient and treatment of the second comorbid health disorder of the patient, the personalized treatment guidance comprising:
a pharmacotherapy component identifying an ordered listing of pharmacological treatment options, each pharmacological treatment option corresponding to a different pharmaceutical product for treatment of the primary health disorder, wherein ordering of the pharmacological treatment options in the ordered listing is based on a ranking of the pharmacological treatment options for treatment of the primary health disorder based on the patient profile data and the diagnostic guidance for the patient;
instructions for generating an electronic report, the report comprising:
an indication of the diagnostic guidance for the patient;
an indication of the personalized treatment guidance to address treatment of the primary health disorder of the patient and treatment of the second comorbid health disorder of the patient.

39. A non-transitory computer readable storage medium having stored therein instructions, which when executed by one or more processors, cause the one or more processors to perform operations for generating personalized diagnostic and treatment guidance, the operations comprising:
obtaining patient profile data for a patient, the patient profile data comprising a result of at least one evaluation test or interview of the patient;
determining, based on the patient profile data, diagnostic guidance for the patient, the diagnostic guidance identifying a primary health disorder of the patient and a second comorbid health disorder of the patient;
determining, based on the patient profile data and the diagnostic guidance for the patient, personalized treatment guidance to address treatment of the primary health disorder of the patient and treatment of the second comorbid health disorder of the patient, the personalized treatment guidance comprising:
a pharmacotherapy component identifying an ordered listing of pharmacological treatment options, each pharmacological treatment option corresponding to a different pharmaceutical product for treatment of the primary health disorder, wherein ordering of the pharmacological treatment options in the ordered listing is based on a ranking of the pharmacological treatment options for treatment of the primary health disorder based on the patient profile data and the diagnostic guidance for the patient;
generating an electronic report, the report comprising:
an indication of the diagnostic guidance for the patient;
an indication of the personalized treatment guidance to address treatment of the primary health disorder of the patient and the second comorbid health disorder of the patient.
